(19) United States
(12) Patent Application Publication
(10) Pub. No.: US 2019/0160098 A1
(43) Pub. Date: May 30, 2019

(54) CHIMERIC ANTIGEN RECEPTORS AND METHODS OF USE THEREOF

(71) Applicant: DANA-FARBER CANCER INSTITUTE, INC., Boston, MA (US)

(72) Inventor: Carl Novina, Newton, MA (US)

(73) Assignee: DANA-FARBER CANCER INSTITUTE, INC., Boston, MA (US)

(21) Appl. No.: 16/077,937

(22) PCT Filed: Feb. 16, 2017
(86) PCT No.: PCT/US17/18188

§ 371 Date: Aug. 14, 2018

Related U.S. Application Data
(60) Provisional application No. 62/295,884, filed on Feb. 16, 2016.

(51) Int. Cl. A61K 35/17; A61K 38/17; A61P 37/06; A61P 35/00; C12N 5/0783; C12N 15/62; C12N 15/85

(52) U.S. Cl. CPC A61K 35/17; A61K 38/1774; A61P 37/06; A61K 2035/122; C12N 5/0636; C12N 15/62; C12N 15/85; A61P 35/00

(57) ABSTRACT

The present invention provides chimeric antigen receptors, cells expressing same and methods of using same for treatment various disorders such as cancer, autoimmune disorders and graft vs host disease.

### RELATED APPLICATIONS

This application claims priority to, and the benefit of U.S. Provisional Application No. 62/295,884 filed on Feb. 16, 2016 and the content of which is incorporated herein by reference in its entirety.

### GOVERNMENT INTEREST

This invention was made with government support under CA185151-02 awarded by the National Cancer Institute and DK105602-01 awarded by the National Institute of Heath. The government has certain rights in the invention.

### FIELD OF THE INVENTION

The present invention relates generally to personalized chimeric antigen receptor cells for and methods of using same for treatment cancer and other disorders.

### INCORPORATION BY REFERENCE OF SEQUENCE LISTING

The contents of the text file name “DFCI-127-001 WO_ST25.txt,” which was created on Jan. 17, 2017 and is 93 KB in size, are hereby incorporated by reference in their entirety.

### BACKGROUND OF THE INVENTION

T lymphocytes recognize specific antigens through interaction of the T cell receptor (TCR) with short peptides presented by major histocompatibility complex (MHC) class I or II molecules. For initial activation and clonal expansion, naive T cells are dependent on professional antigen-presenting cells (APCs) that provide additional co-stimulatory signals. TCR activation in the absence of co-stimulation can result in unresponsiveness and clonal anergy. To bypass immunization, different approaches for the derivation of cytotoxic effector cells with grafted recognition specificity have been developed. Chimeric antigen receptors (CARs) have been constructed that consist of binding domains derived from natural ligands or antibodies specific for cell-surface antigens, genetically fused to effector molecules such as the TCR alpha and beta chains, or components of the TCR-associated CD3 complex. Upon antigen binding, such chimeric antigen receptors link to endogenous signaling pathways in the effector cell and generate activating signals similar to those initiated by the TCR complex. Since the first reports on chimeric antigen receptors, this concept has steadily been refined and the molecular design of chimeric receptors has been optimized.

The most clinically successful CARs use antibodies directed against the CD19 which demonstrates a very narrow expression window (i.e., only on B cells and no other cell types). These CARs are effective because all these B cell-derived malignancies express the B cell specific CD19 surface protein. However, there has been very little success in CAR T cell therapy for solid tumors, as it is difficult to identifying antigens that are present on tumors and not on normal cells. The current invention solves this problem.

### SUMMARY OF THE INVENTION

In various aspects the invention provides a chimeric antigen receptor (CAR) having an intracellular signaling domain, a transmembrane domain and an extracellular domain.

In various aspects the invention provides a chimeric antigen receptor (CAR) for use in treating a subject in need thereof. The CAR includes an intracellular signaling domain, a transmembrane domain and an extracellular domain. The extracellular domain includes i) the variable region of a T-cell receptor specific for a tumor-associated antigen or ii) the variable region of a T-cell receptor specific for a self antigen. The T-cell receptor is derived from a T-lymphocyte obtained from the subject.

The transmembrane domain further comprises a stalk region positioned between the extracellular domain and the transmembrane domain. The transmembrane domain includes CD28.

The CAR further includes one or more additional costimulatory molecules positioned between the transmembrane domain and the intracellular signaling domain. The costimulatory molecules is for example, CD28, 4-1BB, 4-1BBL ICOS, or OX40.

In various aspects the intracellular signaling domain comprises a CD3 zeta chain.

Also provided is a nucleic acid encoding the CAR according to the invention, vectors including the nucleic acid and cells containing the vector. The cell is a T cell such as for example, a CD4⁺ T-cell and/or CD8⁺ T-cell a T regulatory cell (Treg) or a T follicular regulatory cell (TFR).

In other aspects the invention provides a genetically engineered cell which expresses and bears on the cell surface membrane the chimeric antigen receptor according to the invention. The cell is a T cell such as for example, a CD4⁺ T-cell and/or CD8⁺ T-cell a T regulatory cell (Treg) or a T follicular regulatory cell (TFR).

In other aspects the invention provides a pharmaceutical composition containing the population of the genetically engineered cells according to the invention.

In yet a further aspect, the invention provides methods of treating cancer in a subject in need thereof comprising administering the composition of the inventions wherein the extracellular domain of the CAR is a variable region of a T-cell receptor specific for a tumor-associated antigen derived from the subject.

In another aspect, the invention provides methods of treating or preventing an autoimmune disorder in a subject in need thereof comprising administering the composition of the invention, wherein the extracellular domain of the CAR is a variable region of a T-cell receptor specific for a self antigen derived from the subject.

In yet another aspect, the invention provides methods of treating or preventing graft rejection in a subject in need thereof comprising administering the composition of the invention, wherein the extracellular domain of the CAR is a variable region of a T-cell receptor specific for a self antigen derived from the graft tissue.

In further aspects the invention provides methods of isolating a subject specific neoantigen by isolating a plurality of class I and class II MHC peptides from a population of cells obtained from said subject; contacting the plurality of peptide isolated with a T-cell receptor (TCR) multimer, where the TCR was isolated from a single T-cell isolated from the subject to form an TCR multimer-MHC peptide complex; and isolating the MHC peptide from the complex. Optionally, the method further includes sequencing the isolated MHC peptide to identify the peptide. Isolation of the peptides is accomplished immunochemically. The T-cell is a tumor infiltrating lymphocyte, lymphocyte isolated from a transplanted organ or a lymphocyte isolated from an autoimmune site.

In a further aspect the invention provides a vaccine composition including neoantigen peptides identified by the methods of the invention.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1. Is a schematic of RQ-101a, an auto-reactive autologous regulatory T cell, expressing recombinant TCRs derived from single, clonally amplified auto-reactive T cells.

FIG. 2. Is a schematic of RQ-101b, a tumor-specific autologous T cell expressing recombinant TCRs derived from single, clonally amplified Tumor infiltrating lymphocytes (TILs).

FIG. 3. Illustrates the autologous cell manufacturing process.

FIG. 4. Is a schematic of the manufacturing process.

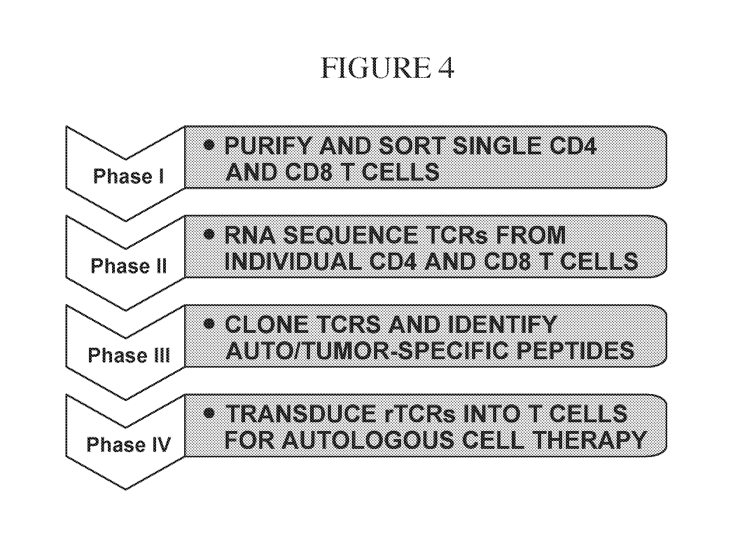

FIG. 5. Illustrates phase 1 of the manufacturing process, the isolation of CD4 and CD8 T cells from tumors. FACS sort into 384 well plates for single cell sequencing.

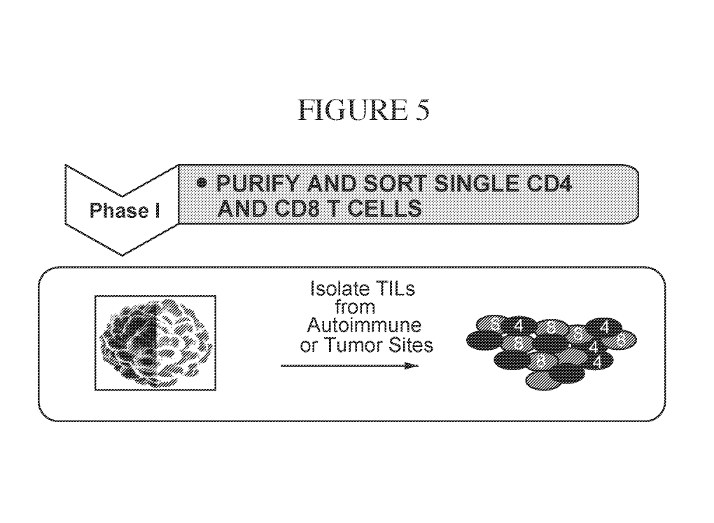

FIG. 6. Illustrates phase 2 of the manufacturing process, the clonally amplified T cells expressing TCRs that react with auto/tumor antigens.

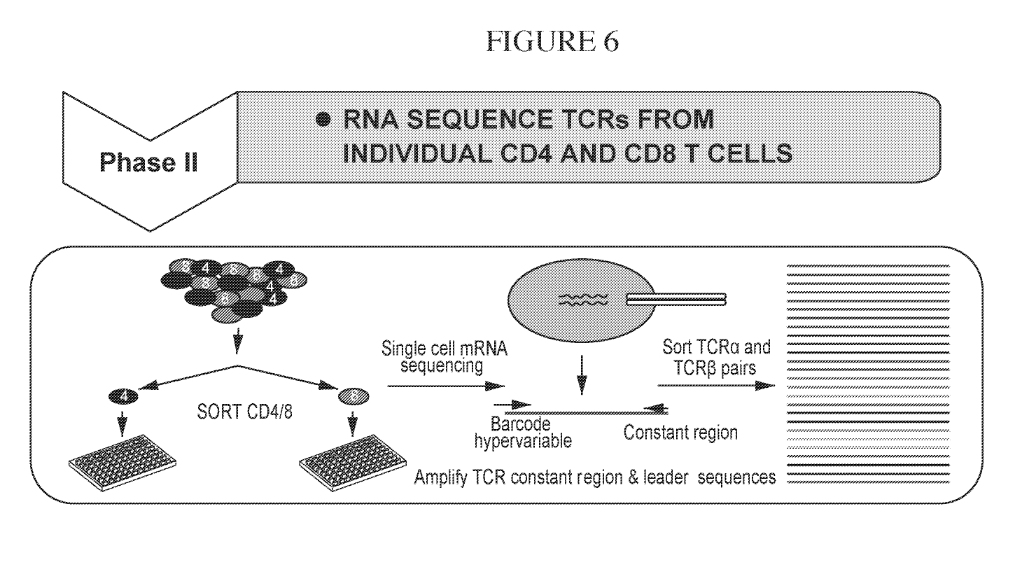

FIG. 7. Illustrates phase 3 of the manufacturing process, the recombinant TCRs that are cloned into CD4 and CD8 T cells. These constructs express naturally occurring TCRs or chimeric TCRs.

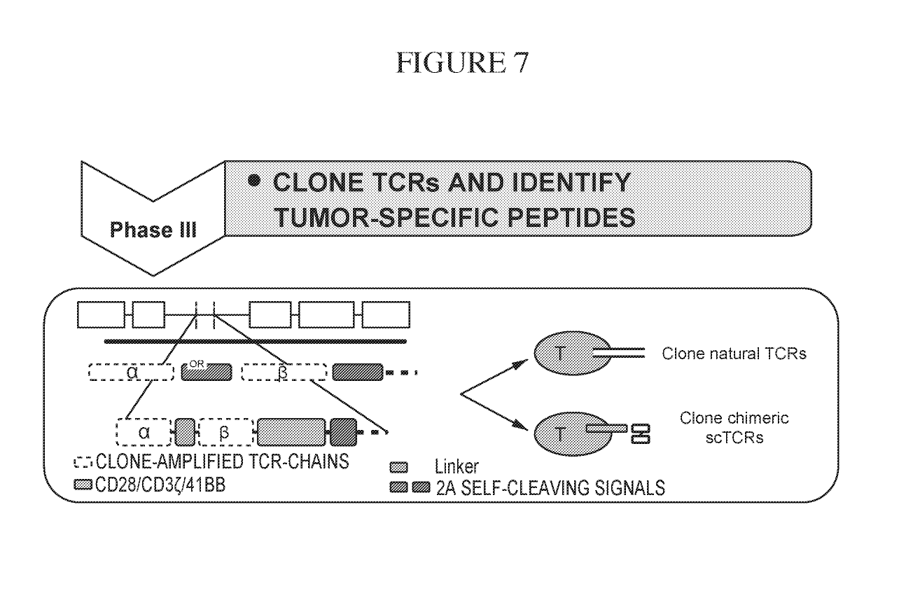

FIG. 8. Illustrates phase 3 of the manufacturing process, the recombinant TCRs that are coupled to beads for purification of peptide-MHC complexes. Immunoprecipitates are subjected to mass spectrometry to identify peptides and alignment to reference genomes to identifies expressed auto/neo-antigens.

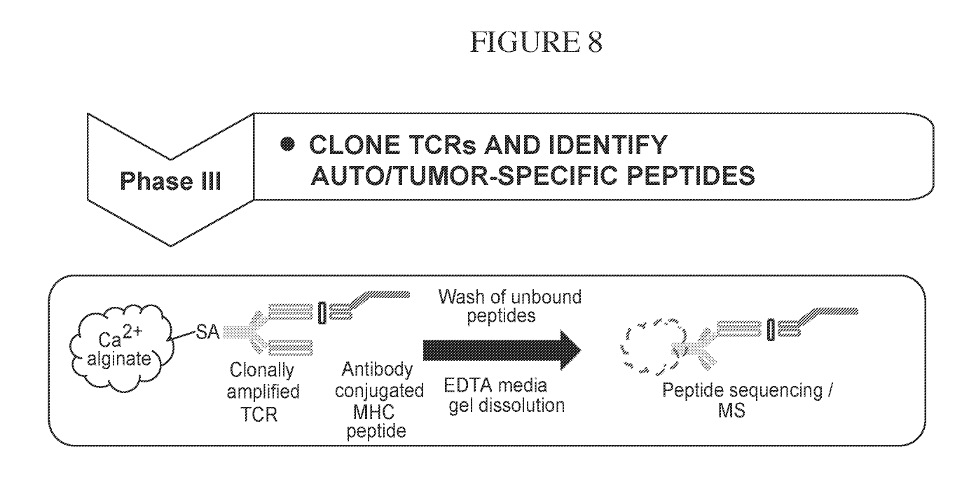

FIG. 9. Is a schematic illustrating phase 4 of the manufacturing process, the in-vitro and ex-vivo killing or suppression assay before transferring the CAR T cells back into patients.

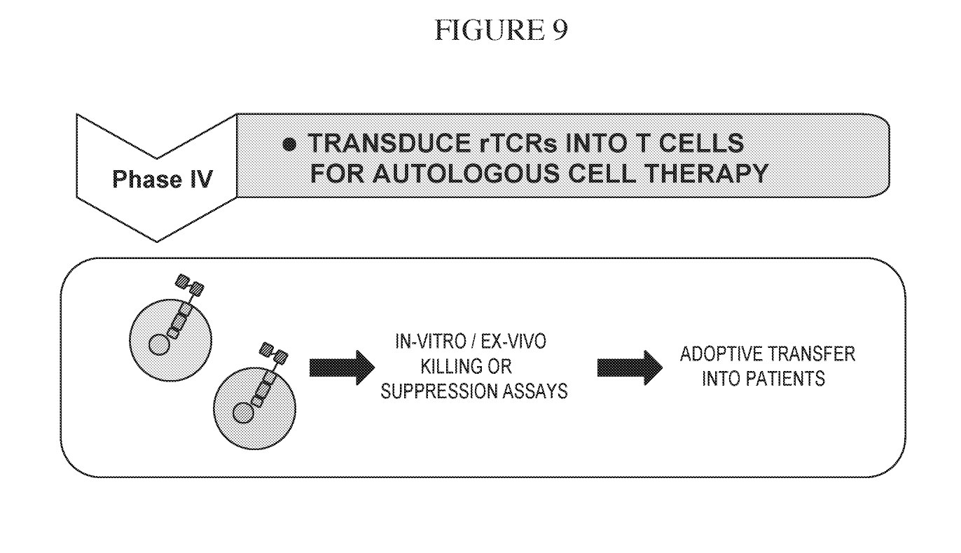

FIG. 10. Schematic representation of the changes that will be applied on Vignali's retroviral vector. LEFT. Representation of the construct that will be used to obtain cytotoxicity in CD8. RIGHT. Representation of the construct used to obtain transplant acceptance in TREG.

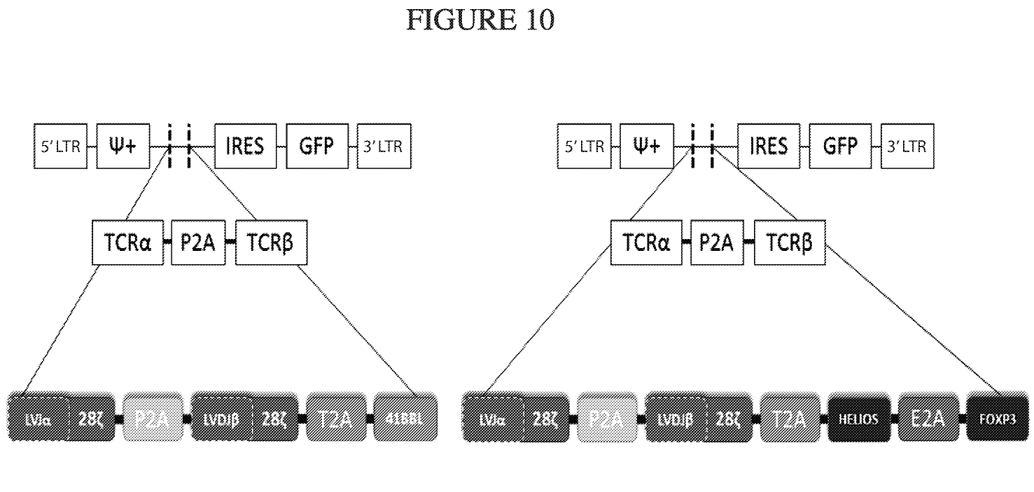

FIG. 11. Schematic representation of the strategy that will be followed to obtain the two different plasmid for retroviral infection.

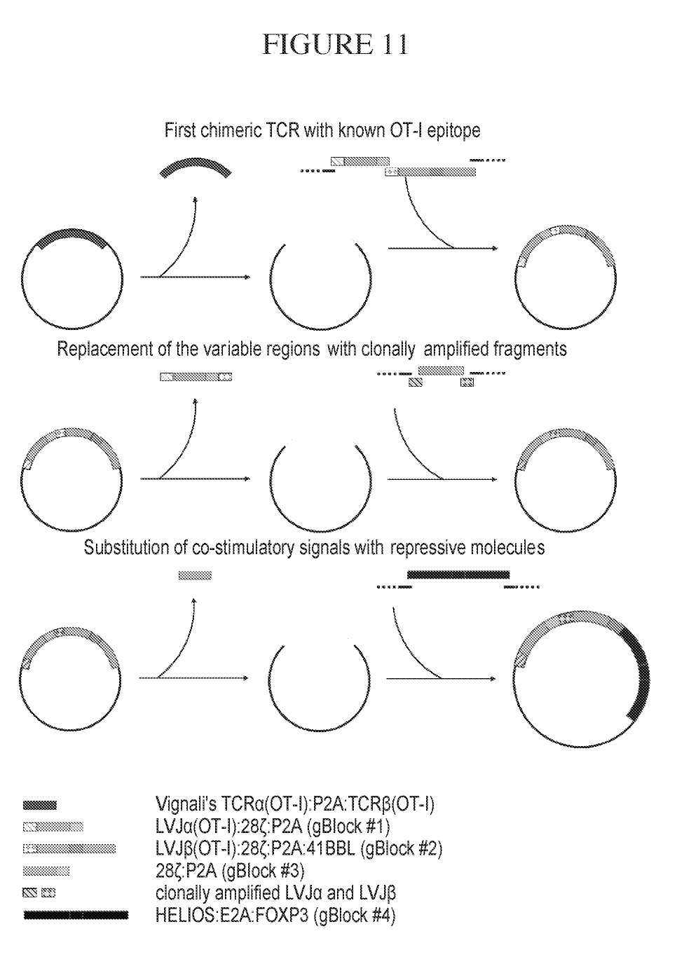

### DETAILED DESCRIPTION OF THE INVENTION

The present invention relates to a chimeric antigen receptor (CAR) particularly adapted to immune cells used in immunotherapy to treat cancer, autoimmune disease and graft vs host disease.

Recently, a series of clinical trials have demonstrated that cancer immunotherapies, can induce durable responses in patients with advanced cancers. One of the most successful cancer immunotherapies uses chimeric antigen receptor (CAR) T cells to treat B cell derived leukemias and lymphomas. In this therapy, a single chain antibody specific for CD19 is fused to CD28 (a T cell co-stimulatory protein) and to CD3zeta. T cells expressing this CD19-specific CAR can provide T cell with potent primary and secondary signals directed against all cells expressing CD19, including malignant and normal B cells. The success of these therapies depends on maximizing immune responses against malignant cells and minimizing immune responses on against normal cells (i.e., off target effects).

To date, CAR T cell therapies have had modest success against solid tumors, in part, because it is difficult to identify antigens that are expressed only on cancer cells but not on normal cells. Unlike CD19 which is expressed uniquely in normal and malignant B cells, there are no other known proteins expressed in solid tumors demonstrate a very narrow window of expression as CD19. Moreover, sequencing has revealed that the majority of tumors generate private mutations not frequently shared by patients with the same tumor type. Indeed, rapidly generating patient-specific immunotherapies is an unmet medical need in not only cancer but in autoimmune disease and graft vs host disease.

The present invention solves the problems of current CAR T cell therapies.

When T cells enter tumors they typically begin to proliferate but are then blocked from further growth by the tumor microenvironment. In the present invention, tumor-infiltrating lymphocytes (TILs) are isolated and single cell targeted RNA-sequencing (RNA-seq) of their TCRs are performed.

TCRs that react to antigens expressed in the tumor by counting the frequency of specific TCRα and β pairs in the individual cells. In tumor cells, since the T cells began to clonally amplify within the tumor prior to becoming inhibited, these TCRs are reacting to antigens in the tumor. TCRs (full-length TCRs or the variable region genes of TCR α/βchains (only) are cloned into a CAR constructs (for example but not limited to variable domains coupled to CD28 and CD3zeta co-stimulatory domains) used to infect peripheral T cells, and administered back into the patient as patient-tumor-specific autologous T cell immunotherapy.

Expressing these recombinant TCRs or CAR-TCRs in effector T cells can generate personalized autologous T cell-based cancer therapy.

To discern which TCRs recognize self antigens and which recognize unique non-self (neo-antigens) in the tumor, we will immunoaffinity purify peptide-MHC complexes using antibodies to MHC Class I and Class II antigens. Then we will perform immunoaffinity purification using TCR tetramers generated from the cloned TCR α/β pairs identified by single cell RNA-seq. Precipitates from tandem immunoaffinity purification will be subjected to mass spectrometry. By comparing peptides to reference genes, we will identify TCRs that recognize peptide neoantigens that uniquely react to the tumor and that do not react to normal healthy tissues. This rapid and direct strategy to identify tumor-specific antigens are used for autologous T cell therapy according to the invention as well as to generate patient-specific peptide vaccines.

Similar to the tumor microenvironment, T cells clonally amplify in other disorders such as autoimmunity and graft (transplant) rejection. Performing single cell targeted RNA-seq of TCRs from sites of autoimmune reactivity and graft rejection can determine the identity and frequency of TCR α/β pairs that are promoting disease. Expressing these recombinant TCRs or CAR-TCRs in regulatory T cells and other regulatory immune cells can generate personalized T cell-based therapy for autoimmunity and graft rejection.

Genetic engineering of human lymphocytes to express tumor-directed or self-directed chimeric antigen receptors (CAR) can produce effector cells that bypass immune escape mechanisms that are due to abnormalities in protein-antigen processing and presentation.

CAR T cell therapies have received much attention, however this immunotherapeutic approach has its limitations. One shortcoming with this strategy is that targeting tumor antigens sometimes comes at the expense of normal cells that are expressing the same proteins, resulting in devastating side effects. In the case of CAR T cells directed against leukemias and lymphomas, this includes an immune attack on patients' normal B cells resulting in the loss of protective antibodies and an increased susceptibility to infections. Moreover identifying tumor antigens is difficult. Second, T cells that enter into the tumor microenvironment are subjected to several inhibitory signals which limit an effective immune response.

The present invention overcomes these challenges by leveraging several technological innovations to create an effective and personalized CAR T cell therapy. Specifically, tumor infiltrating tumor-infiltrating lymphocytes (TILs) will be isolated and the natural T cell receptors (TCRs) on these TILs will be identified and sequenced on a single cell level. The antigens recognized by the natural TCRs will be sequences to determine if they are self-antigens (expressed on normal tissues) or neo-antigens (expressed only on cancer tissues). To make CAR T cells for cancer immunotherapy, CD4⁺ and/or CD8⁺ T-cells will be engineered to express TCRs recognizing only the neo-antigens but not self-antigens. Optionally, to further enhance the CAR T cells' ability to kill tumors, the cells will be further engineered to turn off genes that inhibit immune responses. By turning off the inhibitors of immune responses (such as Cbl-b, SOCS1 and PD-1) only in CAR T cells that recognize cancers, we will vastly improve the anti-cancer effects of CAR T cells without harming normal tissues.

A similar approach is used to produce CAR T cell therapy for autoimmunity and graft vs host disease. Specifically, T-lymphocytes that infiltrate graft tissue or the site of autoimmune reactivity will be isolated and the natural T cell receptors (TCRs) on these TILs will be identified and sequenced on a single cell level. To make CAR T cells for autoimmunity and graft vs host disease, T regulatory cells (Treg) will be engineered to express the identified TCRs.

Also included in the invention are methods of identifying the neoantigen recognized by the isolated TCR. The neoantigen is identified by first isolating MHC class 1 and MHC class II peptides from a cell from the subject. For example, MHC peptides are pulled down using pan-class I and pan class 11 antibodies. Isolated peptides that bind the TCR will be identified by binding to TCR tetramers. The precipitates from this second pulldown will be subjected to mass spectrometry in order to identify the peptide sequences.

The CAR according to the invention generally comprises at least one transmembrane polypeptide comprising at least one extracellular ligand-binding domain and; one transmembrane polypeptide comprising at least one intracellular signaling domain; such that the polypeptides assemble together to form a Chimeric Antigen Receptor.

The term “extracellular ligand-binding domain” as used herein is defined as an polypeptide that is capable of binding a ligand. Preferably, the domain will be capable of interacting with a cell surface molecule. For example, the extracellular ligand-binding domain may be chosen to recognize a ligand that acts as a cell surface marker on target cells associated with a particular disease state. Most preferably, the extracellular domain is the variable region of a T-cell receptor. Most preferably, the extracellular domain is a variable region of a T-cell receptor that recognize a tumor antigen.

In particular, the extracellular ligand-binding domain comprises the variable region of a T-cell receptor specific for a tumor associated antigen or a self antigen. Preferably, the T-cell receptor, the tumor associated antigen or self antigen has been identified from a single T-cell obtained from a subject. For example, the T-cell receptor is identified from a tumor infiltrating lymphocyte, a lymphocyte from an autoimmune site or a lymphocyte from a graft tissue.

In various aspects the extracellular ligand-binding domain is a single chain T-cell receptor.

In a preferred embodiment said transmembrane domain further comprises a stalk region between said extracellular ligand-binding domain and said transmembrane domain. The term “stalk region” used herein generally means any oligo- or polypeptide that functions to link the transmembrane domain to the extracellular ligand-binding domain. In particular, stalk region are used to provide more flexibility and accessibility for the extracellular ligand-binding domain. A stalk region may comprise up to 300 amino acids, preferably 10 to 100 amino acids, more preferably 25 to 50 amino acids and most preferably 3 to 15 amino acids. Stalk region may be derived from all or part of naturally occurring molecules, such as from all or part of the extracellular region of CD8, CD4 or CD28, or from all or part of an antibody constant region. Alternatively the stalk region may be a synthetic sequence that corresponds to a naturally occurring stalk sequence, or may be an entirely synthetic stalk sequence. In a preferred embodiment said stalk region is a part of human CD8 alpha chain

The signal transducing domain or intracellular signaling domain of the CAR of the invention is responsible for intracellular signaling following the binding of extracellular ligand binding domain to the target resulting in the activation of the immune cell and immune response. In other words, the signal transducing domain is responsible for the activation of at least one of the normal effector functions of the immune cell in which the CAR is expressed. For example, the effector function of a T cell can be a cytolytic activity or helper activity including the secretion of cytokines. Thus, the term “signal transducing domain” refers to the portion of a protein which transduces the effector signal function signal and directs the cell to perform a specialized function.

Signal transduction domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Examples of ITAM used in the invention can include as non limiting examples those derived from TCR zeta. FcR gamma, FcR beta, FcR epsilon, CD3 gamma, CD3 delta. CD3 epsilon, CD5, CD22, CD79a, CD79b and CD66d. In a preferred embodiment, the signaling transducing domain of the CAR can comprise the CD3 zeta signaling domain, or the intracytoplasmic domain of the Fc epsilon RI beta or gamma chains. In another preferred embodiment, the signaling is provided by CD3 zeta together with co-stimulation provided by CD28 and a tumor necrosis factor receptor (TNFr), such as 4-1BB or OX40), for example.

In particular embodiment the intracellular signaling domain of the CAR of the present invention comprises a co-stimulatory signal molecule. In some embodiments the intracellular signaling domain contains 2, 3, 4 or more co-stimulatory molecules in tandem. A co-stimulatory molecule is a cell surface molecule other than an antigen receptor or their ligands that is required for an efficient immune response.

“Co-stimulatory ligand” refers to a molecule on an antigen presenting cell that specifically binds a cognate co-stimulatory molecule on a T-cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM, CD30L, CD40. CD70, CD83, HLA-G, MICA, M1CB, HVEM, lymphotoxin beta receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27. CD28, 4-1BB, OX40. CD30, CD40. PD-1, ICOS, lymphocyte function-associated antigen-1 (LFA-1), CD2, CD7, LTGHT, NKG2C, B7-H3, a ligand that specifically binds with CD83.

A “co-stimulatory molecule” refers to the cognate binding partner on a T-cell that specifically binds with a co-stimulatory ligand, thereby mediating a co-stimulatory response by the cell, such as, but not limited to proliferation. Co-stimulatory molecules include, but are not limited to an MHC class I molecule, BTLA and Toll ligand receptor. Examples of costimulatory molecules include CD3, CD27, CD28, CD8, 4-1BB (CD137). OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like.

In another particular embodiment, said signal transducing domain is a TNFR-associated Factor 2 (TRAF2) binding motifs, intracytoplasmic tail of costimulatory TNFR member family. Cytoplasmic tail of costimulatory TNFR family member contains TRAF2 binding motifs consisting of the major conserved motif (P/S/A)X(Q/E)E) or the minor motif (PXQXXD), wherein X is any amino acid. TRAF proteins are recruited to the intracellular tails of many TNFRs in response to receptor trimerization.

The distinguishing features of appropriate transmembrane polypeptides comprise the ability to be expressed at the surface of an immune cell, in particular lymphocyte cells or Natural killer (NK) cells, and to interact together for directing cellular response of immune cell against a predefined target cell. The different transmembrane polypeptides of the CAR of the present invention comprising an extracellular ligand-biding domain and/or a signal transducing domain interact together to take part in signal transduction following the binding with a target ligand and induce an immune response. The transmembrane domain can be derived either from a natural or from a synthetic source. The transmembrane domain can be derived from any membrane-bound or transmembrane protein.

The term “a part of” used herein refers to any subset of the molecule, that is a shorter peptide. Alternatively, amino acid sequence functional variants of the polypeptide can be prepared by mutations in the DNA which encodes the polypeptide. Such variants or functional variants include, for example, deletions from, or insertions or substitutions of, residues within the amino acid sequence. Any combination of deletion, insertion, and substitution may also be made to arrive at the final construct, provided that the final construct possesses the desired activity, especially to exhibit a specific anti-target cellular immune activity. The functionality of the CAR of the invention within a host cell is detectable in an assay suitable for demonstrating the signaling potential of said CAR upon binding of a particular target. Such assays are available to the skilled person in the art. For example, this assay allows the detection of a signaling pathway, triggered upon binding of the target, such as an assay involving measurement of the increase of calcium ion release, intracellular tyrosine phosphorylation, inositol phosphate turnover, or interleukin (IL) 2, interferon .gamma., GM-CSF, IL-3, IL-4 production thus effected.

Cells

Embodiments of the invention include cells that express a CAR (i.e, CARTS). The cell may be of any kind, including an immune cell capable of expressing the CAR for cancer therapy or a cell, such as a bacterial cell, that harbors an expression vector that encodes the CAR. As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a eukaryotic cell that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid. In embodiments of the invention, a host cell is a T cell, including a helper T cell (Th), a cytotoxic T cell (also known as TC, Cytotoxic T Lymphocyte, CTL, T-Killer cell, cytolytic T cell, CD8+ T-cells or killer T cell) a regulatory T cell (Treg), a T follicular regulatory cell (TFR), NK cells and NKT cells are also encompassed in the invention.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

The cells can be autologous cells, syngeneic cells, allogenic cells and even in some cases, xenogeneic cells.

In many situations one may wish to be able to kill the modified CTLs, where one wishes to terminate the treatment, the cells become neoplastic, in research where the absence of the cells after their presence is of interest, or other event. For this purpose one can provide for the expression of certain gene products in which one can kill the modified cells under controlled conditions, such as inducible suicide genes.

Armed CARTS

The invention further includes CARTS that are modified to secrete one or more polypeptides. The polypeptide can be for example an antibody or cytokine. Cytokines included for example IL-2.

Armed CARTS have the advantage of simultaneously secreting a polypeptide at the targeted site, e.g. tumor site, graft site or autoimmune site.

Armed CART can be constructed by including a nucleic acid encoding the polypeptide of interest after the intracellular signaling domain. Preferably, there is an internal ribosome entry site, (IRES), positioned between the intracellular signaling domain and the polypeptide of interest. One skilled in the art can appreciate that more than one polypeptide can be expressed by employing multiple IRES sequences in tandem.

Introduction of Constructs into Cells

Expression vectors that encode the CARs can be introduced as one or more DNA molecules or constructs, where there may be at least one marker that will allow for selection of host cells that contain the construct(s).

The constructs can be prepared in conventional ways, where the genes and regulatory regions may be isolated, as appropriate, ligated, cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagenesis, etc., as appropriate. The construct(s) once completed and demonstrated to have the appropriate sequences may then be introduced into the cell (i.e., T-cell) by any convenient means. The constructs may be integrated and packaged into non-replicating, defective viral genomes like Adenovirus, Adeno-associated virus (AAV), or Herpes simplex virus (HSV) or others, including retroviral vectors or lentiviral vectors, for infection or transduction into cells. The constructs may include viral sequences for transfection, if desired. Alternatively, the construct may be introduced by fusion, electroporation, biolistics, transfection, lipofection, or the like. The host cells may be grown and expanded in culture before introduction of the construct(s), followed by the appropriate treatment for introduction of the construct(s) and integration of the construct(s). The cells are then expanded and screened by virtue of a marker present in the construct. Various markers that may be used successfully include hprt, neomycin resistance, thymidine kinase, hygromycin resistance, etc.

In some instances, one may have a target site for homologous recombination, where it is desired that a construct be integrated at a particular locus. For example,) can knock-out an endogenous gene and replace it (at the same locus or elsewhere) with the gene encoded for by the construct using materials and methods as are known in the art for homologous recombination. For homologous recombination, one may use either .OMEGA. or O-vectors. See, for example, Thomas and Capecchi, Cell (1987) 51, 503-512; Mansour, et al., Nature (1988) 336, 348-352; and Joyner, et al., Nature (1989) 338, 153-156.

The constructs may be introduced as a single DNA molecule encoding at least the CAR and optionally another gene, or different DNA molecules having one or more genes. Other genes include genes that encode therapeutic molecules or suicide genes, for example. The constructs may be introduced simultaneously or consecutively, each with the same or different markers.

Vectors containing useful elements such as bacterial or yeast origins of replication, selectable and/or amplifiable markers, promoter/enhancer elements for expression in prokaryotes or eukaryotes, etc. that may be used to prepare stocks of construct DNAs and for carrying out transfections are well known in the art, and many are commercially available.

Methods of Use

The cells according to the invention can be used for treating cancer, graft vs host disease or autoimmune disorders in a patient in need thereof. In another embodiment, said isolated cell according to the invention can be used in the manufacture of a medicament for treatment of a cancer, graft vs host disease or autoimmune disorders, in a patient in need thereof.

The present invention relies on methods for treating patients in need thereof, said method comprising at least one of the following steps: (a) providing a chimeric antigen receptor cells according to the invention and (b) administrating the cells to said patient.

Said treatment can be ameliorating, curative or prophylactic. It may be either part of an autologous immunotherapy or part of an allogenic immunotherapy treatment. By autologous, it is meant that cells, cell line or population of cells used for treating patients are originating from said patient or from a Human Leucocyte Antigen (HLA) compatible donor. By allogeneic is meant that the cells or population of cells used for treating patients are not originating from said patient but from a donor.

The invention is particularly suited for allogenic immunotherapy, insofar as it enables the transformation of T-cells, typically obtained from donors, into non-alloreactive cells. This may be done under standard protocols and reproduced as many times as needed. The resulted modified T cells may be pooled and administrated to one or several patients, being made available as an “off the shelf” therapeutic product.

Cells that can be used with the disclosed methods are described in the previous section. Said treatment can be used to treat patients diagnosed with cancer, autoimmune disorders or Graft versus Host Disease (GvHD). Cancers that may be treated include tumors that are not vascularized, or not yet substantially vascularized, as well as vascularized tumors. The cancers may comprise nonsolid tumors (such as hematological tumors, for example, leukemias and lymphomas) or may comprise solid tumors. Types of cancers to be treated with the CARs of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

It can be a treatment in combination with one or more therapies against cancer selected from the group of antibodies therapy, chemotherapy, cytokines therapy, dendritic cell therapy, gene therapy, hormone therapy, laser light therapy and radiation therapy.

According to a preferred embodiment of the invention, said treatment can be administrated into patients undergoing an immunosuppressive treatment. Indeed, the present invention preferably relies on cells or population of cells, which have been made resistant to at least one immunosuppressive agent due to the inactivation of a gene encoding a receptor for such immunosuppressive agent. In this aspect, the immunosuppressive treatment should help the selection and expansion of the T-cells according to the invention within the patient.

In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAM PATH. In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery. Said modified cells obtained by any one of the methods described here can be used in a particular aspect of the invention for treating patients in need thereof against Host versus Graft (HvG) rejection and Graft versus Host Disease (GvHD); therefore in the scope of the present invention is a method of treating patients in need thereof against Host versus Graft (HvG) rejection and Graft versus Host Disease (GvHD) comprising treating said patient by administering to said patient an effective amount of modified cells comprising inactivated TCR alpha and/or TCR beta genes.

Administration of Cells

The invention is particularly suited for allogenic immunotherapy, insofar as it enables the transformation of T-cells, typically obtained from donors, into non-alloreactive cells. This may be done under standard protocols and reproduced as many times as needed. The resulted modified T cells may be pooled and administrated to one or several patients, being made available as an “off the shelf” therapeutic product.

Depending upon the nature of the cells, the cells may be introduced into a host organism, e.g. a mammal, in a wide variety of ways. The cells may be introduced at the site of the tumor, in specific embodiments, although in alternative embodiments the cells hone to the cancer or are modified to hone to the cancer. The number of cells that are employed will depend upon a number of circumstances, the purpose for the introduction, the lifetime of the cells, the protocol to be used, for example, the number of administrations, the ability of the cells to multiply, the stability of the recombinant construct, and the like. The cells may be applied as a dispersion, generally being injected at or near the site of interest. The cells may be in a physiologically-acceptable medium.

In some embodiments, the cells are encapsulated to inhibit immune recognition and placed at the site of the tumor.

The cells may be administered as desired. Depending upon the response desired, the manner of administration, the life of the cells, the number of cells present, various protocols may be employed. The number of administrations will depend upon the factors described above at least in part.

The administration of the cells or population of cells according to the present invention may be carried out in any convenient manner, including by aerosol inhalation, injection, ingestion, transfusion, implantation or transplantation. The compositions described herein may be administered to a patient subcutaneously, intradermaly, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. The cells or population of cells can be administrated in one or more doses. In another embodiment, said effective amount of cells are administrated as a single dose. In another embodiment, said effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

It should be appreciated that the system is subject to many variables, such as the cellular response to the ligand, the efficiency of expression and, as appropriate, the level of secretion, the activity of the expression product, the particular need of the patient, which may vary with time and circumstances, the rate of loss of the cellular activity as a result of loss of cells or expression activity of individual cells, and the like. Therefore, it is expected that for each individual patient, even if there were universal cells which could be administered to the population at large, each patient would be monitored for the proper dosage for the individual, and such practices of monitoring a patient are routine in the art.

Nucleic Acid-Based Expression Systems

The CARs of the present invention may be expressed from an expression vector. Recombinant techniques to generate such expression vectors are well known in the art.

Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30 110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5 prime′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

The identity of tissue-specific promoters or elements, as well as assays to characterize their activity, is well known to those of skill in the art.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals

In certain embodiments of the invention, the use of internal ribosome entry sites (IRES) elements are used to create multigene, or polycistronic, messages, and these may be used in the invention.

In other embodiments of the invention, the use of 2A self cleaving peptides are used to create multigene, or polycistronic, messages, and these may be used in the invention.

Vectors can include a multiple cloning site (MCS), which is a nucleic acid region that contains multiple restriction enzyme sites, any of which can be used in conjunction with standard recombinant technology to digest the vector. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Splicing sites, termination signals, origins of replication, and selectable markers may also be employed.

Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™ 11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S transferase (GST) soluble fusion proteins for later purification and separation or cleavage. Other suitable fusion proteins are those with galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

Viral Vectors

The ability of certain viruses to infect cells or enter cells via receptor mediated endocytosis, and to integrate into host cell genome and express viral genes stably and efficiently have made them attractive candidates for the transfer of foreign nucleic acids into cells (e.g., mammalian cells). Components of the present invention may be a viral vector that encodes one or more CARs of the invention. Non-limiting examples of virus vectors that may be used to deliver a nucleic acid of the present invention are described below.

Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell specific construct that has been cloned therein. Knowledge of the genetic organization or adenovirus, a 36 kb, linear, double stranded DNA virus, allows substitution of large pieces of adenoviral DNA with foreign sequences up to 7 kb (Grunhaus and Horwitz, 1992).

AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno associated virus (AAV) is an attractive vector system for use in the cells of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

Retroviral Vectors

Retroviruses are useful as delivery vectors because of their ability to integrate their genes into the host genome, transferring a large amount of foreign genetic material, infecting a broad spectrum of species and cell types and of being packaged in special cell lines (Miller, 1992).

In order to construct a retroviral vector, a nucleic acid (e.g., one encoding the desired sequence) is inserted into the viral genome in the place of certain viral sequences to produce a virus that is replication defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

Vector Delivery and Cell Transformation

Suitable methods for nucleic acid delivery for transfection or transformation of cells are known to one of ordinary skill in the art. Such methods include, but are not limited to, direct delivery of DNA, RNA or mRNA such as by ex vivo transfection, by injection, and so forth. Through the application of techniques known in the art, cells may be stably or transiently transformed.

Ex Vivo Transformation

Methods for transfecting eukaryotic cells and tissues removed from an organism in an ex vivo setting are known to those of skill in the art. Thus, it is contemplated that cells or tissues may be removed and transfected ex vivo using nucleic acids of the present invention. In particular aspects, the transplanted cells or tissues may be placed into an organism. In preferred facets, a nucleic acid is expressed in the transplanted cells.

### Kits of the Invention

Any of the compositions described herein may be comprised in a kit. In a non-limiting example, one or more cells for use in cell therapy and/or the reagents to generate one or more cells for use in cell therapy that harbors recombinant expression vectors may be comprised in a kit. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile, pharmaceutically acceptable buffer and/or other diluent.

In particular embodiments of the invention, cells that are to be used for cell therapy are provided in a kit, and in some cases the cells are essentially the sole component of the kit. The kit may comprise reagents and materials to make the desired cell. In specific embodiments, the reagents and materials include primers for amplifying desired sequences, nucleotides, suitable buffers or buffer reagents, salt, and so forth, and in some cases the reagents include vectors and/or DNA that encodes a CAR as described herein and/or regulatory elements therefor.

In particular embodiments, there are one or more apparatuses in the kit suitable for extracting one or more samples from an individual. The apparatus may be a syringe, scalpel, and so forth.

In some cases of the invention, the kit, in addition to cell therapy embodiments, also includes a second cancer therapy, such as chemotherapy, hormone therapy, and/or immunotherapy, for example. The kit(s) may be tailored to a particular cancer for an individual and comprise respective second cancer therapies for the individual.

Identification of Neoantigens

One of the critical barriers to developing curative and tumor-specific immunotherapy is the identification and selection of highly restricted tumor antigens to avoid autoimmunity. Tumor neoantigens, which arise as a result of genetic change within malignant cells, represent the most tumor-specific class of antigens. Neoantigens have rarely been used in vaccines due to technical difficulties in identifying them. Our approach to identify tumor-specific neoantigen involves. (1) identification of T cell receptor sequences that react with antigens expressed in the tumor obtained from the patient patient; (2) cloning the patient specific T cell receptors; (3) isolating class I and class II peptides from the patient; (4) contacting the MHC peptides with the TCR to (5) identifying what MHC peptides for a complex with the T cell receptor.

The MHC peptides (neoantigen peptides) are identified for example by direct protein sequencing. Protein sequencing of enzymatic digests using multidimensional MS techniques (MSn) including tandem mass spectrometry (MS/MS)) can also be used to identify neoantigens of the invention. Such proteomic approaches permit rapid, highly automated analysis (see, e.g., K. Gevaert and J. Vandekerckhove, Electrophoresis 21:1145-1154 (2000)).

Accordingly, the present invention also provides to methods for identifying and/or detecting T-cell epitopes of an antigen. Specifically, the invention provides method of identifying and/or detecting tumor specific neoantigens that are useful in inducing a tumor specific immune response in a subject.

The invention further includes the isolated peptides that comprise the neoantigen identified by the methods of the invention. The size of the neoantigenic peptide molecule may comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein. In specific embodiments the neoantigenic peptide molecules are equal to or less than 50 amino acids. In some embodiments the particular neoantigenic peptides and polypeptides of the invention are: for MHC Class I 13 residues or less in length and usually consist of between about 8 and about 11 residues, particularly 9 or 10 residues; for MHC Class II, 15-24 residues. The neoantigenic peptides and polypeptides bind an HLA protein. The neoantigenic peptide has an IC50 of at least less than 5000 nM, at least less than 500 nM, at least less then 250 nM, at least less than 200 nM, at least less than 150 nM, at least less than 100 nM, at least less than 50 nM or less. The neoantigenic peptides and polypeptides does not induce an autoimmune response and/or invoke immunological tolerance when administered to a subject.

One of skill in the art from this disclosure and the knowledge in the art will appreciate that there are a variety of ways in which to produce such tumor specific neoantigens suitable for administration as a vaccine to a patient. In general, such tumor specific neoantigens may be produced either in vitro or in vivo. Tumor specific neoantigens may be produced in vitro as peptides or polypeptides, which may then be formulated into a personalized neoplasia vaccine or immunogenic composition and administered to a subject. Such in vitro production may occur by a variety of methods known to one of skill in the art such as, for example, peptide synthesis or expression of a peptide/polypeptide from a DNA or RNA molecule in any of a variety of bacterial, eukaryotic, or viral recombinant expression systems, followed by purification of the expressed peptide/polypeptide. Alternatively, tumor specific neoantigens may be produced in vivo by introducing molecules (e.g., DNA, RNA, viral expression systems, and the like) that encode tumor specific neoantigens into a subject, whereupon the encoded tumor specific neoantigens are expressed.

The invention further provides a method of vaccinating or treating a subject by administering the neoantigen peptides identified by the methods of the invention to the subject.

The vaccine composition can further comprise an adjuvant and/or a carrier. The peptides and/or polypeptides in the composition can be associated with a carrier such as e.g. a protein or an antigen-presenting cell such as e.g. a dendritic cell (DC) capable of presenting the peptide to a T-cell.

Adjuvants are any substance whose admixture into the vaccine composition increases or otherwise modifies the immune response to the mutant peptide. Carriers are scaffold structures, for example a polypeptide or a polysaccharide, to which the neoantigenic peptides, is capable of being associated. Optionally, adjuvants are conjugated covalently or non-covalently to the peptides or polypeptides of the invention.

The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th response into a primarily cellular, or Th response.

Suitable adjuvants include, but are not limited to 1018 ISS, aluminium salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V. Montanide ISA-51, OK-432, OM-174, OM-197-MP-EC, ONTAK, PepTel® vector system, PLG microparticles, resiquimod, SRL 172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants such as incomplete Freund's or GM-CSF are preferred. Several immunological adjuvants (e.g., MF59) specific for dendritic cells and their preparation have been described previously (Dupuis M, et al., Cell Immunol. 1998; 186(1):18-27; Allison A C. Dev Biol Stand. 1998; 92:3-11). Also cytokines may be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418).

CpG immunostimulatory oligonucleotides have also been reported to enhance the effects of adjuvants in a vaccine setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly, it enhances dendritic cell maturation and differentiation, resulting in enhanced activation of TH1 cells and strong cytotoxic T-lymphocyte (CTL) generation, even in the absence of CD4 T-cell help. The TH1 bias induced by TLR9 stimulation is maintained even in the presence of vaccine adjuvants such as alum or incomplete Freund's adjuvant (IFA) that normally promote a TH2 bias. CpG oligonucleotides show even greater adjuvant activity when formulated or co-administered with other adjuvants or in formulations such as microparticles, nano particles, lipid emulsions or similar formulations, which are especially necessary for inducing a strong response when the antigen is relatively weak. They also accelerate the immune response and enabled the antigen doses to be reduced by approximately two orders of magnitude, with comparable antibody responses to the full-dose vaccine without CpG in some experiments (Arthur M. Krieg, Nature Reviews, Drug Discovery, 5, Jun. 2006, 471-484). U.S. Pat. No. 6,406,705 BI describes the combined use of CpG oligonucleotides, non-nucleic acid adjuvants and an antigen to induce an antigen-specific immune response. A commercially available CpG TLR9 antagonist is dSLIM (double Stem Loop Immunomodulator) by Mologen (Berlin. GERMANY), which is a preferred component of the pharmaceutical composition of the present invention. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly(I:C) (e.g. polyi:CI2U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives useful in the context of the present invention can readily be determined by the skilled artisan without undue experimentation. Additional adjuvants include colony-stimulating factors, such as Granulocyte Macrophage Colony Stimulating Factor (GM-CSF, sargramostim).

A carrier may be present independently of an adjuvant. The function of a carrier can for example be to increase the molecular weight of in particular mutant in order to increase their activity or immunogenicity, to confer stability, to increase the biological activity, or to increase serum half-life. Furthermore, a carrier may aid presenting peptides to T-cells. The carrier may be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier must be a physiologically acceptable carrier acceptable to humans and safe. However, tetanus toxoid and/or diptheria toxoid are suitable carriers in one embodiment of the invention. Alternatively, the carrier may be dextrans for example sepharose.

Combination Therapy

In certain embodiments of the invention, methods of the present invention for clinical aspects are combined with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cancer cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with other therapies. In the context of the present invention, it is contemplated that cell therapy could be used similarly in conjunction with chemotherapeutic, radiotherapeutic, or immunotherapeutic intervention, as well as pro-apoptotic or cell cycle regulating agents.

Alternatively, the present inventive therapy may precede or follow the other agent treatment by intervals ranging from minutes to weeks. In embodiments where the other agent and present invention are applied separately to the individual, one would generally ensure that a significant period of time did not expire between the time of each delivery, such that the agent and inventive therapy would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

It is expected that the treatment cycles would be repeated as necessary. It also is contemplated that various standard therapies, as well as surgical intervention, may be applied in combination with the inventive cell therapy.

Chemotherapy

Cancer therapies also include a variety of combination therapies with both chemical and radiation based treatments. Combination chemotherapies include, for example, abraxane, altretamine, docetaxel, herceptin, methotrexate, novantrone, zoladex, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, famesyl-protein tansferase inhibitors, transplatinum, 5-fluorouracil, vincristin, vinblastin and methotrexate, or any analog or derivative variant of the foregoing and also combinations thereof.

In specific embodiments, chemotherapy for the individual is employed in conjunction with the invention, for example before, during and/or after administration of the invention

Radiotherapy

Other factors that cause DNA damage and have been used extensively include what are commonly known as .gamma-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

Immunotherapy

Immunotherapeutics generally rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy other than the inventive therapy described herein could thus be used as part of a combined therapy, in conjunction with the present cell therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include PD-1, PD-L1, CTLA4, carcinoembryonic antigen, prostate specific antigen, urinary tumor associated antigen, fetal antigen, tvrosinase (p97), gp68, TAG-72, HMFG, Sialyl Lewis Antigen, MucA, MucB, PLAP, estrogen receptor, laminin receptor, erb B and p155.

Genes

In yet another embodiment, the secondary treatment is a gene therapy in which a therapeutic polynucleotide is administered before, after, or at the same time as the present invention clinical embodiments. A variety of expression products are encompassed within the invention, including inducers of cellular proliferation, inhibitors of cellular proliferation, or regulators of programmed cell death.

Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/RAIL would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

### EXAMPLES

### Example 1: Cloning T Cell Receptors from Single Tumor Infiltrating Lymphocytes (TILs) for Cancer Immunotherapy

Phase 1: Purification and Sorting of Single CD4 and CD8 T Cells from Tumors

The goal of this phase is to isolate CD4 and CD8 T cells from tumors. The central idea of this phase is to recover as many CD4 and CD8 TILs as possible. Identification and cloning of individual CD4 TCRs can provide T cell help against antigens in tumors. Identification and cloning of individual CD8 TCRs can provide antigen-specific T cell killing of tumors.

Phase 2: Sequencing TCRs RNA from CD4 AND CD8 TILs

The goal of this phase is to array and sequence individual CD4 and CD8 T cells from tumors. The central idea is that T cells that proliferate in the tumor are reacting to antigens present in tumors. At single cell resolution, the T cell receptors that are clonally amplified are likely to be tumor reactive as compared to individual T cells that might happen to be present in blood vessels or near a tumor but not reacting against tumor cells.

We have developed a novel TCR sequencing strategy for single cells. Unlike current methods, our approach is unbiased and does not require complex primer sets against the variable region:

TCRα and TCRβ variable regions will be reverse transcribed using an oligo dT primer and an MMLV strand switching reverse transcriptase. This enzyme appends a universal primer site to the 3′ end of the first strand cDNA via the strand switching mechanism, thus allowing amplification of all TCR cDNA molecules using only the universal primer and a nested seed primer complimentary to the constant region.

cDNAs will be barcoded during amplification, which enables pooling of hundreds of cells in a single sequencing reaction and marks TCRa/b reads from individual cells with the same barcode. Identification of α/β pairing is critical because antigen recognition depends on the formation of a specific α/β dimer.

We will perform Illumina paired end sequencing with 300 base reads. Illumina is the most cost-effective and efficient platform for providing deep coverage of TCR variable regions in many cells. However, if 300 base reads are insufficiently long to cover the entire variable region, we will use the Pacific Biosystems platform which produces reads that average over 10 kilobases in length.

Phase 3: Cloning TCRs into Retroviral Expression Constructs

The goal of this phase is to design a universal vector that allows cloning of recombinant TCRs into CD4 and CD8 T cells. These constructs allow transduction of T cells with naturally occurring TCRs or with engineered chimeric antigen receptor (CAR) T cell receptors specific for tumors.

The retroviral vector we have chosen for proof of concept is based on the vector described by Hoist et al., which allows stoichiometric expression of TCRα and TCRβ in multi-cistronic vectors. Expression of the single proteins is enabled by the 2A auto-cleaving signals. We will clone this construct by Gibson assembly. To improve TCR signaling and T cell proliferation, the variable portion TCRs that recognizes peptide-MHC is fused in cis to the fusion chimera CD28/CD3ζ. This CAR design in a T cell receptor context has not been previously reported. We anticipate that this context will increase T cells' cytotoxicity and proliferation at tumor sites. Chimeric TCRs reduce the chance that recombinant TCRs will pair with endogenous TCRs expressed in transduced T cells. We will express natural and chimeric TCRs in both CD4 and CD8 T cells.

Phase 4: Ex Vivo and In Vivo Testing for Recombinant TCRs

The goal of this phase is to express CD4 and CD8 TCRs cloned from individual TILs in T cells. The central idea of this phase is “personalized” TCR therapy against specific tumors. We anticipate that clonally amplified TCRs react to antigens present in tumors. The TCRs that amplify in the tumor react against tumor antigens. These will be tested for activity in an in vitro killing assay. However, it is also possible that TCRs cloned from TILs can cross-react with non-tumor (self) antigens. To reduce the chances that TCRs cloned from TILs cross-react with self (non-tumor) peptides, we will perform an in vitro killing assay using normal cells from the tissue that led to the tumor.

In vitro killing assay: T cells will be mixed with 4×103 tumor cells (lymphoma B cells) or normal B cells from genetically matched mice at various effector:target ratios in U-bottom 96-well plates, spun at 200 rpm for 2 min before being transferred into incubator. After 4 hr incubation, cultures will be stained with antibodies for CD19 and active Caspase-3 (BD), and analyzed by FACS. Active Caspase-3 positive CD19+ cells represent apoptotic target cells. % specific killing=% apoptotic target cells of cultures with both effectors and targets−% apoptotic target cells of cultures with targets alone. This assay is easier and safer than traditional chromium release assay and has been well accepted in the field.

In vivo tumor protection: 1×10⁴ tumor cells will be transferred (i.v.) into Rag2−/—γc−/− mice either alone or together with 1×10⁵ T cells. Recipients will be sacrificed on day 30 after transplantation to examine tumor sizes.

### Example 2: Cloning T Cell Receptors from Single Infiltrating Lymphocytes from Autoimmune Sites and Transplanted Organs

Phase 1: Purification and Sorting of Single CD4 and CD8 T Cells from Inflammatory Sites or Transplanted Organs

The goal of this phase is to isolate populations CD4 and CD8 T cells from sites of autoimmune reactivity or from transplanted organs. The central idea of this phase is to recover as many auto-reactive CD4 and CD8 cells or host CD4 and CD8 cells that infiltrate the donor organ as possible. Individual TCRs that have clonally amplified in these sites can identify the relevant TCR sequences that when expressed in suppressor T cells can generate patient-specific suppression of T cells reacting against antigens in sites of auto-immune reactivity or transplanted organs.

Phase 2: Sequencing TCRs RNA from CD4 AND CD8 Lymphocytes from Auto-Immune Sites or Transplanted Organs

The goal of this phase is to array and sequence individual CD4 and CD8 T cells infiltrating sites of autoimmune reactivity and transplanted organs. The central idea is that T cells infiltrating these sites react against antigens relevant to autoimmunity or transplant rejection. The clonally amplified TCRs react against self peptides or donor peptides and can be distinguished from random individual T cells that might happen to be present in blood vessels which do not react against self or donor peptides

We have developed a novel TCR sequencing strategy for single cells. Unlike current methods, our approach is unbiased and does not require complex primer sets against the variable region:

TCRα and TCRβ variable regions will be reverse transcribed using an oligo dT primer and an MMLV strand switching reverse transcriptase. This enzyme appends a universal primer site to the 3′ end of the first strand cDNA via the strand switching mechanism, thus allowing amplification of all TCR cDNA molecules using only the universal primer and a nested seed primer complimentary to the constant region.

cDNAs will be barcoded during amplification, which enables pooling of hundreds of cells in a single sequencing reaction and marks TCRa/b reads from individual cells with the same barcode. Identification of α/β pairing is critical because antigen recognition depends on the formation of a specific α/β dimer.

We will perform Illumina paired end sequencing with 300) base reads. Illumina is the most cost-effective and efficient platform for providing deep coverage of TCR variable regions in many cells. However, if 300 base reads are insufficiently long to cover the entire variable region, we will use the Pacific Biosystems platform which produces reads that average over 10 kilobases in length.

Phase 3: Cloning TCRS into Retroviral Expression Constructs

The goal of this phase is to design a universal vector that allows cloning of CD4 and CD8 TCRs that were identified from tissues with autoimmune reactive T cells or from transplanted organs with donor-specific T cells. Sequences from clonally amplified TCRs reacting against self peptides or donor peptides will be inserted into constructs that allow transduction of CD4 and CD8 T regulatory cells (TREGs) and other immune inhibitory cells. Recombinant TCRs can be naturally occurring TCRs or engineered chimeric antigen receptor (CAR) T cell receptors specific for self or donor antigens. This strategy will enable personalized TCR immunotherapy to repress self- or donor-reactive T cells in an antigen specific fashion.

The retroviral vector we have chosen for proof of concept is based on the vector described by Holst et al., which allows stoichiometric expression of TCRα and TCRβ in multi-cistronic vectors. Expression of the single proteins is enabled by the 2A auto-cleaving signals. We will clone this construct by Gibson assembly. To improve TCR signaling and T cell proliferation, the variable portion TCRs that recognizes peptide-MHC is fused in cis to the fusion chimera CD28/CD3ζ. This CAR design in a T cell receptor context has not been previously reported. We anticipate that this context will increase T cells' cytotoxicity and proliferation at tumor sites. Chimeric TCRs reduce the chance that recombinant TCRs will pair with endogenous TCRs expressed in transduced T cells. We will express natural and chimeric TCRs in both CD4 and CD8 T cells.

Phase 4: Ex Vivo and In Vivo Testing for Recombinant TCRs

The goal of this phase is to express CD4 and CD8 TCRs cloned from individual T cells infiltrating autoimmune sites or graft rejection sites. The central idea of this phase is “personalize” TCR therapy by reducing function of auto-reactive T cells or host cells directed against graft antigens. We anticipate that clonally amplified TCRs that react against self or donor antigens can be ectopically expressed in immune suppressor cells such as TREGs, induced TREG (iTREGs), and T follicular regulatory cells (TFR). These T cells will be tested for suppressor activity in an in vitro assay and in vivo in mouse models of cartilage-induced autoimmunity. To demonstrate antigen specific suppression by Treg expressing recombinant TCRs, antigen presenting cells (APC, prepared from spleens) will be incubated with synovial tissue lysate to load APC with peptides. Activated T cells isolated from synovial tissue will be incubated with Treg expressing recombinant TCRs which are expected suppress activated T cells.

We will express natural and chimeric TCRs in CD4 Treg, CD8 Treg, and in Tfr.

We will express natural and chimeric TCRs in iTregs. This will be accomplished by expressing either FOXP3 and/or Helios in CD4 and CD8 effector cells.

We will perform in vitro TREG suppression assay using a protocol in which suppression of anti-CD3/CD28 activated T cell will be incubated with TREG expressing recombinant TCR and measuring the level of activated T cell proliferation.

Although Tregs can suppress immune responses in an antigen-non-specific fashion, Tregs suppress ˜1000 fold more efficiently in an antigen-specific fashion. Thus, the Treg suppression assay will determine sensitivity of antigen-specific Treg-mediated suppression. The OT-II TCR transgenic system will be used as a proof-of-concept for the efficiency of antigen-specific Treg suppression. In this system, using OT-II-peptide coated APC in vitro, we will measure the level of suppression by polyclonal Treg and compare this level to the level of suppression by Tregs expressing the OT-II TCR.

### Example 3: General TCR Cloning Strategy

Choice of the Vector

A retroviral vector was chosen because of the existence of a publication (Holst et al., 2006), which focused on the expression of TCR-α and TCR-β. The authors use a single vector to express simultaneously both TCR chains. This expression system presents some advantages: (1) the system works with any mouse strain and it was already applied to test TCRs in vivo; (2) tt is faster (6 weeks vs. 6 months) than making transgenic mice; (3) he expression of TCR-α and TCR-β is stoichiometric; (4) the vectors are available at Addgene. Disadvantages of the system include: (1) the 2A tag remains on the C-terminus of the protein located upstream of its sequence. Nevertheless, the authors do not observe alterations in the TCR's expression or function and (2) T cells develop in adult mice with a memory-like phenotype.

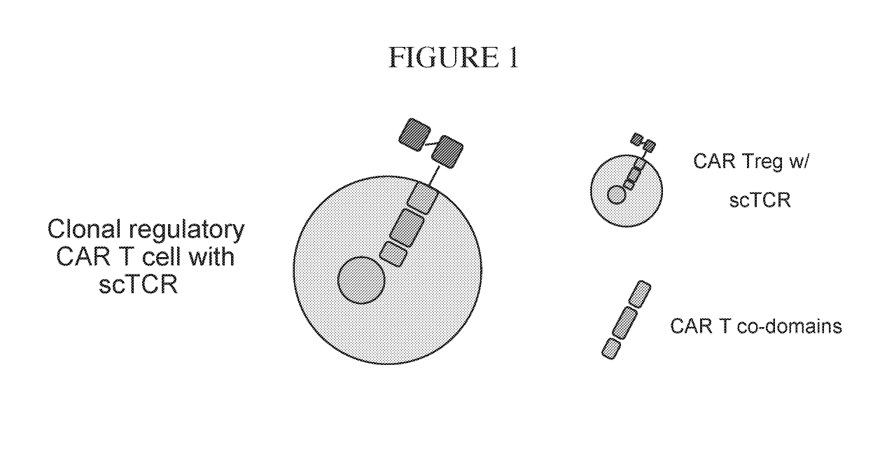

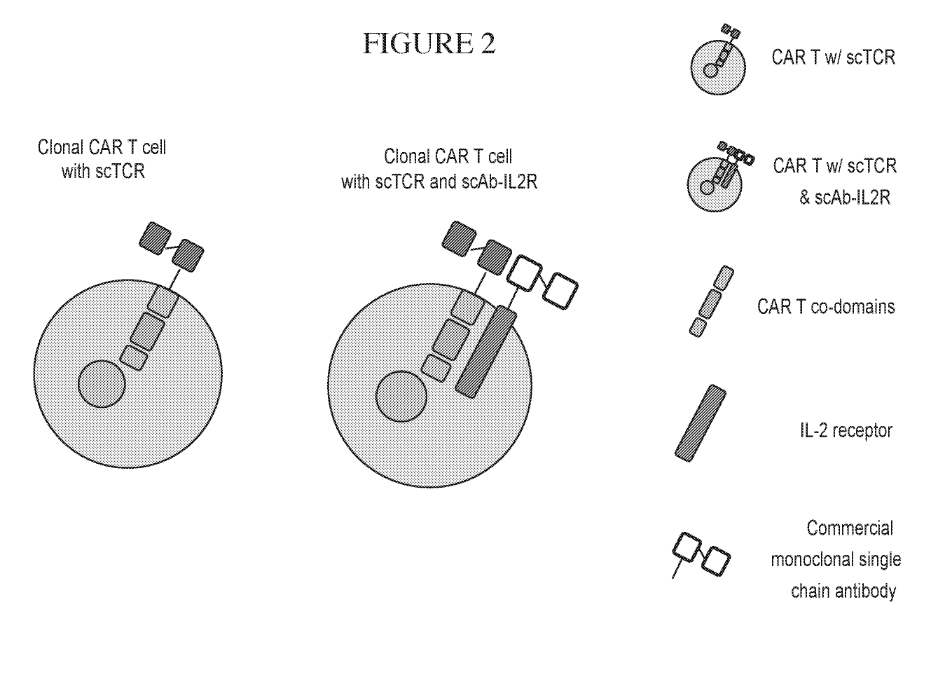

The publication is connected to two different constructs in the Addgene database: murine TCR OTI-2A.pMIG II (#52111) and murine TCR OTII-2A.pMIG II (#52112) and the former will be used as scaffold. In particular, in order to respect the correct positioning of the elements to be included the ORF2 containing Vignali's TCRs will be completely substituted by our construct (FIGS. 1 and 2). (Host et al., 2006)

Finally, the Vignali groups reports that “because retroviruses are known to recombine vector sequences that contain duplications of homologous regions . . . different 2A peptide sequences with silent mutations were used within the constructs containing three or four 2A peptide-linked cistrons”. Therefore, the same design was reproduced at the plasmid level (Szymczak et al., 2004): the α and the β chains share the CD28:CD3 co-stimulatory domain sequences. o reduce the chances of recombination, the nucleotides that codify for the CD28:CD3 on the TCRP were manually chosen to generate a fragment the most possible diverse from the one that codifies for the same residues on the TCRα chain.

To perform the expression of four different proteins through a multi-cistronic vector it is required the use of maximum three different autocleaving 2A sequences. Kim and coworkers compare four different cleaning signals which seem to have different cleaving efficiency. Among the four presented, P2A, T2A and E2A are the most active (Kim et al., 2011).

Vector Template Design

Plasmid #52111 translated sequence (frame 2) was compared with the sequences found in the IMGT database. The correct expression of TCRα and TCRβ chain was ensured by using the elements shown in Table 1. This design will be used as template for the construction of our vector. All the sequences that are used as template are compatible to our host (C57BL/6×BALB/c)F1 mouse (http://www.imgt.or/vquest/refseqh.html).

| Segment | Length | Amino-acid sequence |
|---|---|---|
| TCRα-L | 28 | MDKILTASFLLLGLHLAGVNGQQQEKRD (SEQ ID NO: 1) |
| TCRα-V | 93 | QQQVRQSPQSLTVWEGETAILNCSYEDSTFNYFPWYQQFPGEG |
| | | PALLISIRSVSDKKEDGRFTIFFNKREKKLSLHITDSQPGDSATYF |
| | | CAAS (SEQ ID NO: 2) |
| TCRα-J | 17 | DNYQLIWGSGTKLIIKP (SEQ ID NO: 55) |
| TCRα-C | 135 | DIQNPEPAVYQLKDPRSQDSTLCLFTDFDSQINVPKTMESGTFIT |
| | | DKTVLDMEAMDSKSNGAIAWSNQTSFTCQDIFKETNATYPSSD |
| | | VPCDATLTEKSFETDMNLNFQNLSVMGLRILLLKVAGFNLLMT |
| | | LRLW (SEQ ID NO: 3) |
| SPACER | 3 | GSG |
| P2A | 19 | ATNFSLLKQAGDVEENPG\|\|P (SEQ ID NO: 4) |
| TCRβ-L | 29 | MSNTVLADSAWGITLLSWVTVFLLGTSSA (SEQ ID NO: 5) |
| TCRβ-V | 94 | DSGVVQSPRHIIKEKGGRSVLTCIPISGHSNVVWYQQTLGKELK |
| | | FLIQHYEKVERDKGFLPSRFSVQQFDDYHSEMNMSALELEDSA |
| | | MYFCASS (SEQ ID NO: 6) |
| TCRβ-D | 2 | RA (deduced-not in IMGT) |
| TCRβ-J | 15 | NYEQYFGPGTRLTVL (SEQ ID NO: 7) |
| TCRβ-C | 173 | EDLRNVTPPKVSLFEPSKAEIANKQKATLVCLARGFFPDHVELS |
| | | WWVNGKEVHSGVSTDPQAYKESNYSYCLSSRLRVSATFWHNP |
| | | RNHFRCQVQFHGLSEEDKWPEGSPKPVTQNISAEAWGRADCGI |
| | | TSASYHQGVLSATILYEILLGKATLYAVLVSGLVLMAMVKKKN |
| | | S* (SEQ ID NO: 8) |

Elements to be Included in the Vector

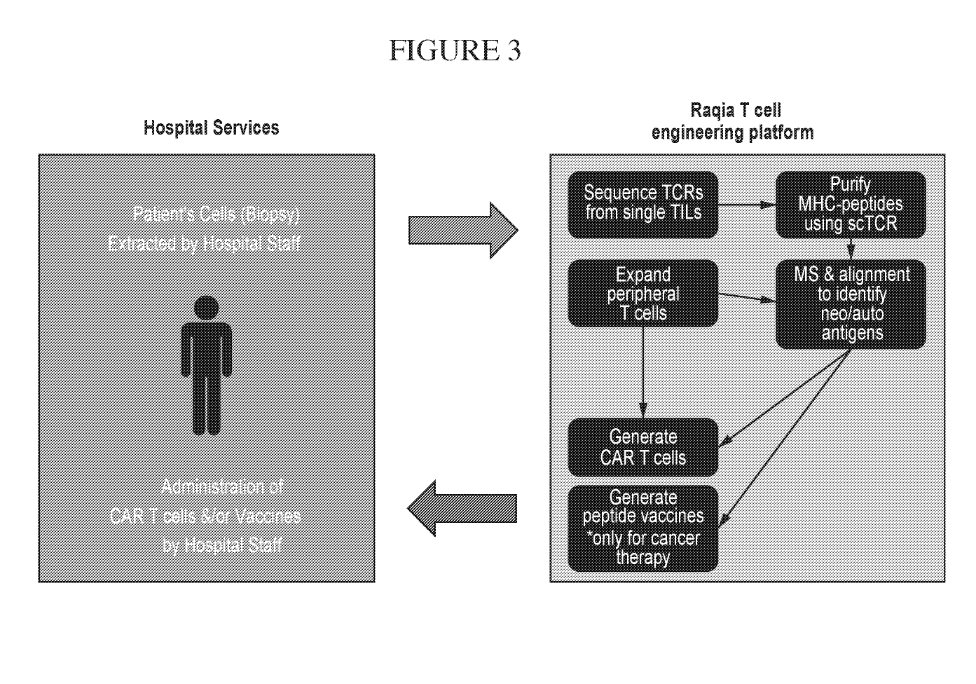

The vector will include the following entities in the following order (See, FIG. 3):
[LVJ]α28:3ζ
(GSG)P2A
[LVDJ]β:28:3ζ
(GSG)T2A
4-1BBL
(HELIOS)
((GSG)E2A)
(FOXP3)

Template Sequences

The Sadelain group reports the sequences of CD28 and CD3ζ used to clone a human chimeric TCR in a recent publication (Maher et al., 2002). To define the homologous regions of these proteins in mouse the sequences were aligned with the help of a standard alignment tool (blastP https://blast.ncbi.nlm.nih.gov/). The seed sequences are shown below in the FASTA format. The homologous regions were used for the constructs.

The part to be described in the literature is highlighted in yellow and the parts that will be cloned are highlighted in green (Maher et al., 2002; Sadelain et al., 2004). Unfortunately, the majority of the publications do not include a detailed description of the chimeric protein, therefore, no more recent publication could be used as reference.

CD28_mouse (UniProtKB-P31041)
Human template
>sp|P10747|CD28_HUMA T-cell-specific surface glycoprotein CD28
OS = Homo sapiens GN = CD28 PE = 1 SV = 1
(SEQ ID NO: 9)
MLRLLLALNLFPSIQVTGNKILVKQSPMLVAYDNAVNLSCKYSYNLFSR
EFRASLHKGLDSAVEVCVVYGNYSQQLQVYSKTGFNCDGKLGNESVTFYLQNLYV
NQTDIYFCKIEVMYPPPYLDNEKSNGTIIHVKGKHLCPSPLFPGPSKPFWVLVVVGG
VLACYSLLVTVAFIIFWVRSKRSRLLHSDYMNMTPRRPGPTRKHYQPYAPPRDFAA
YRS
Mouse analog (106 aa)
>sp|P31041|CD28_MOUSE T-cell-specific surface glycoprotein CD28
OS = Mus musculus GN = Cd28 PE = 2 SV = 2
(SEQ ID NO: 10)
MTLRLLFLALNFFSVQVTENKILVKQSPLLVVDSNEVSLSCRYSYNLLAK
EFRASLYKGVNSDVEVCVGNGNFTYQPQFRSNAEFNCDGDFDNETVTFRLWNLHV
NHTDIYFCKIEFMYPPPYLDNERSNGTIIHIKEKHLCHTQSSPKLFWALVVVAGVLF
CYGLLVTVALCVIWTNSRRNRLLQSDYMNMTPRRPGLTRKPYQPYAPARDFAAYR
P
CD3ζ_mouse (UniProtKB-P24161-1)
Human template
>sp|P20963|CD3Z_HUMAN T-cell surface glycoprotein CD3 zeta chain
OS = Homo sapiens GN = CD247 PE = 1 SV = 2
(SEQ ID NO: 11)
MKWKALFTAAILQAQLPITEAQSFGLLDPKLCYLLDGILFIYGVILTALFL
RVKFSRSADAPAYQQGQNQLYNELNLGRREEYDVLDKRRGRDPEMGGKPQRRKN
PQEGLYNELQKDKMAEAYSEIGMKGERRRGKGHDGLYQGLSTATKDTYDALHMQ
ALPPR
Mouse analog (113 aa)
>sp|P24161|CD3Z_MOUSE T-cell surface glycoprotein CD3 zeta chain
OS = Mus musculus GN = Cd247 PE = 1 SV =1
(SEQ ID NO: 12)
MKWKVSVLACILHVRFPGAEAQSFGLLDPKLCYLLDGILFIYGVIITALY
LRAKFSRSAETAANLQDPNQLYNELNLGRREEYDVLEKKRARDPEMGGKQQRRR
NPQEGVYNALQKDKMAEAYSEIGTKGERRRGKGHDGLYQGLSTATKDTYDALHM
QTLAPR

The following sequences will be cloned completely

4-1BB ligandmouse (UniProtKB-P20334)
Mouse template (256 aa)
>sp|P20334|TNR9_MOUSE Tumor necrosis factor receptor superfamily
member 9 OS = Mus musculus GN = Tnfrsf9 PE = 1 SV = 1
(SEQ ID NO: 13)
MGNNCYNVVVIVLLLVGCEKVGAVQNSCDNCQPGTFCRKYNPVCKSCP
PSTFSSIGGQPNCNICRVCAGYFRFKKFCSSTHNAECECIEGFHCLGPQCTRCEKDCR
PGQELTKQGCKTCSLGTFNDQNGTGVCRPWTNCSLDGRSVLKTGTTEKDVVCGPP
VVSFSPSTTISVTPEGGPGGHSLQVLTLFLALTSALLLALIFITLLFSVLKWIRKKEPHI
FKQPFKKTTGAAQEEDACSCRCPQEEEGGGGGYEL
HELIOS_mouse (UniProtKB-P81183-1)
Mouse template (526 aa)
>sp|P81183|IKZF2_MOUSE Zinc finger protein Helios OS = Mus musculus
GN = lkzf2 PE = 1 SV = 2
(SEQ ID NO: 14)
METDAIDGYITCDNELSPEGEHANMAIDLTSSTPNGQHASPSHMTSTNSV
KLEMQSDEECDRQPLSREDEIRGHDEGSSLEEPLIESSEVADNRKVQDLQGEGGIRL
PNGKLKCDVCGMVCIGPNVLMVHKRSHTGERPFHCNQCGASFTQKGNLLRHIKLH
SGEKPFKCPFCSYACRRRDALTGHLRTHSVGKPHKCNYCGRSYKQRSSLEEHKERC
HNYLQNVSMEAAGQVMSHHVPPMEDCKEQEPIMDNNISLVPFERPAVIEKLTANM
GKRKSSTPQKFVGEKLMRFSYPDIHFDMNLTYEKEAELMQSHMMDQAINNAITYL
GAEALHPLMQHAPSTIAEVAPVISSAYSQVYHPNRIERPISRETSDSHENNMDGPISLI
RPKSRPQEREASPSNSCLDSTDSESSHDDRQSYQGNPALNPKRKQSPAYMKEDVKA
LDATKAPKGSLKDIYKVFNGEGEQIRAFKCEHCRVLPLDHVMYTIHMGCHGYRDP
LECNICGYRSQDRYEFSSHIVRGEHTFH
OXP3_mouse (UniProtKB-Q99JB6)
Mouse template (429 aa)
>sp|Q99JB6|FOXP3_MOUSE Forkhead box protein P3 OS = Mus musculus
GN = Foxp3 PE = 1 SV = 1
(SEQ ID NO 15)
MPNPRPAKPMAPSLALGPSPGVLPSWKTAPKGSELLGTRGSGGPFQGRD
LRSGAHTSSSLNPLPPSQLQLPTVPLVMVAPSGARLGPSPHLQALLQDRPHFMHQLS
TVDAHAQTPVLQVRPLDNPAMISLPPPSAATGVFSLKARPGLPPGINVASLEWVSRE
PALLCTFPRSGTPRKDSNLLAAPQGSYPLLANGVCKWPGCEKVFEEPEEFLKHCQA
DHLLDEKGKAQCLLQREVVQSLEQQLELEKEKLGAMQAHLAGKMALAKAPSVAS
MDKSSCCIVATSTQGSVLPAWSAPREAPDGGLFAVRRHLWGSHGNSSFPEFFHNM
DYFKYHNMRPPFTYATLIRWAILEAPERQRTLNEIYHWFTRMFAYFRNHPATWKN
AIRHNLSLHKCFVRVESEKGAVWTVDEFEFRKKRSQRPNKCSNPCP

Documented 2A Autocleaving Sequences

| Name | Length | Sequence |
|---|---|---|
| P2A | 22 aa | GSG ATNFSLLKQAGDVEENPGP |
| | | (SEQ ID NO: 16) |
| T2A | 21 aa | GSG EGRGSLLTCGDVEENPGP |
| | | (SEQ ID NO: 17) |
| E2A | 23 aa | GSG QCTNYALLKLAGDVESNPGP |
| | | (SEQ ID NO: 18) |

### Example 4: TCR Cloning Control

Description of Vignali's Murine TCR OTI-2A.pMIG HI (Plasmid #52111)

TABLE 2
pMIG-OTI (Holst et al., 2006).

| Segment | Length | Amino-acid sequence |
|---|---|---|
| TCRα-L | 28 | MDKILTASFLLLGLHLAGVNGQQQEKRD (SEQ ID NO: 19) |
| TCRα-V | 93 | QQQVRQSPQSLTVWEGETAILNCSYEDSTFNYFPWYQQFPGEG |
| | | PALLISIRSVSDKKEDGRFTIFFNKREKKLSLHITDSQPGDSATYF |
| | | CAAS (SEQ ID NO: 20) |
| TCRα-J | 17 | DNYQLIWGSGTKLIIKP (SEQ ID NO: 21) |
| TCRα-C | 135 | DIQNPEPAVYQLKDPRSQDSTLCLFTDFDSQINVPKTMESGTFIT |
| | | DKTVLDMEAMDSKSNGAIAWSNQTSFTCQDIFKETNATYPSSD |
| | | VPCDATLTEKSFETDMNLNFQNLSVMGLRILLLKVAGFNLLMT |
| | | LRLW (SEQ ID NO:22) |
| SPACER | 3 | GSG |
| P2A | 19 | ATNFSLLKQAGDVEENPG\|\|P (SEQ ID NO: 23) |
| TCRβ-L | 29 | MSNTVLADSAWGITLLSWVTVFLLGTSSA (SEQ ID NO: 24) |
| TCRβ-V | 94 | DSGVVQSPRHIIKEKGGRSVLTCIPISGHSNVVWYQQTLGKELK |
| | | FLIQHYEKVERDKGFLPSRFSVQQFDDYHSEMNMSALELEDSA |
| | | MYFCASS (SEQ ID NO: 25) |
| TCRβ-D | 2 | RA (deduced-not in IMGT) |
| TCRβ-J | 15 | NYEQYFGPGTRLTVL (SEQ ID NO: 26) |
| TCRβ-C | 173 | EDLRNVTPPKVSLFEPSKAEIANKQKATLVCLARGFFPDHVELS |
| | | WWVNGKEVHSGVSTDPQAYKESNYSYCLSSRLRVSATFWHNP |
| | | RNHFRCQVQFHGLSEEDKWPEGSPKPVTQNISAEAWGRADCGI |
| | | TSASYHQGVLSATILYEILLGKATLYAVLVSGLVLMAMVKKKN |
| | | S* (SEQ ID NO: 27) |

TABLE 3
pMIG-OTII (Holst et al., 2006).

| Segment | Length | Amino-acid sequence |
|---|---|---|
| TCRα-L | 28 | MDKILTASFLLLGLHLAGVNGQQQEKRD (SEQ ID NO: 28) |
| TCRα-V | 93 | QQQVRQSPQS...DSATYFCAAR |
| | | (SEQ ID NO: 29) |
| TCRα-J | 16 | GNRIFFGDGTQLVVKP (SEQ ID NO: 30) |
| TCRα-C | 136 | NIQNPEPAVY...NLLMTLRLWS (SEQ ID NO: 31) |
| SPACER | 3 | GSG |
| P2A | 19 | ATNFSLLKQAGDVEENPG\|\|P (SEQ ID NO: 32) |
| TCRβ-L | 29 | MSNTAFPDPAWNTTLLSWVALFLLGTSSA (SEQ ID NO: 33) |
| TCRβ-V | 94 | NSGVVQSPRYI...SAVYFCASSL (SEQ ID NO: 34) |
| TCRβ-D | 2 | GGE (deduced-not in IMGT) |
| TCRβ-J | 16 | SQNTLYFGAGTRLSVL (SEQ ID NO: 35) |
| TCRβ-C | 173 | EDLRNVTPPK...LMAMVKRKNS* (SEQ ID NO: 36) |

TABLE 4
Description of the components that form the vector used by
Vignali and coworkers (Holst et al., 2006).

| Segment | Length | Amino-acid sequence | New/Old |
|---|---|---|---|
| αLVJ | 139 | MDKILTASFLLLGLHLAGVNGQQQEKRDQQQVRQSPQSLTVWEGETAILNCSYEDSTFN | Old |
| | | YFPWYQQFPGEGPALLISIRSVSDKKEDGRFTIFFNKREKKLSLHITDSQPGDSATYFCAAS | |
| | | DNYQLIWGSGTKLIIKPD (SEQ ID NO: 37) | |
| 28ζ | 241 | CKIEFMYPPPYLDNERSNGTIIHIKEKHLCHTQSSPKLFWALVVVAGVLFCYGLLVTVALC | New |
| P2A | | VIWTNSRRNRLLQSDYMNMTPRRPGLTRKPYQPYAPARDFAAYRPRAKFSRSAETAANL | |
| | | QDPNQLYNELNLGRREEYDVLEKKRARDPEMGGKQQRRRNPQEGVYNALQKDKMAEA | |
| | | YSEIGTKGERRRGKGHDGLYQGLSTATKDTYDALHMQTLAPRGSGATNFSLLKQAGDV | |
| | | EENPGP (SEQ ID NO: 38) | |
| βLVDJ | 140 | MSNTVLADSAWGITLLSWVTVFLLGTSSADSGVVQSPRHIIKEKGGRSVLTCIPISGHSNV | Old |
| | | VWYQQTLGKELKFLIQHYEKVERDKGFLPSRFSVQQFDDYHSEMNMSALELEDSAMYF | |
| | | CASSRANYEQYFGPGTRLTVL (SEQ ID NO: 39) | |
| 28 | 496 | CKIEFMYPPPYLDNERSNGTIIHIKEKHLCHTQSSPKLFWALVVVAGVLFCYGLLVTVALC | New |
| ζ | | VIWTNSRRNRLLQSDYMNMTPRRPGLTRKPYQPYAPARDFAAYRPRAKFSRSAETAANL | |
| T2A | | QDPNQLYNELNLGRREEYDVLEKKRARDPEMGGKQQRRRNPQEGVYNALQKDKMAEA | |
| 41BBL | | YSEIGTKGERRRGKGHDGLYQGLSTATKDTYDALHMQTLAPRGSGEGRGSLLTCGDVEE | |
| | | NPGPMGNNCYNVVVIVLLLVGCEKVGAVQNSCDNCQPGTFCRKYNPVCKSCPPSTFSSI | |
| | | GGQPNCNICRVCAGYFRFKKFCSSTHNAECECIEGFHCLGPQCTRCEKDCRPGQELTKQG | |
| | | CKTCSLGTFNDQNGTGVCRPWTNCSLDGRSVLKTGTTEKDVVCGPPVVSFSPSTTISVTP | |
| | | EGGPGGHSLQVLTLFLALTSALLLALIFITLLFSVLKWIRKKFPHIFKQPFKKTTGAAQEED | |
| | | ACSCRCPQEEEGGGGGYEL* (SEQ ID NO: 40) | |

Full construct
MDKILTASFLLLGLHLAGVNGQQQEKRDQQQVRQSPQSLTVWEGETAILNCSYEDSTFNYFPWYQQFPGEGPALLISIRSVSDKKEDG
RFTIFFNKREKKLSLHITDSQPGDSATYFCAASDNYQLIWGSGTKLIIKPDCKIEFMYPPPYLDNERSNGTIIHIKEKHLCHTQSSPKLF
WALVVVAGVLFCYGLLVTVALCVIWTNSRRNRLLQSDYMNMTPRRPGLTRKPYQPYAPARDFAAYRPRAKFSRSAETAANLQDPNQLYNE
LNLGRREEYDVLEKKRARDPEMGGKQQRRRNPQEGVYNALQKDKMAEAYSEIGTKGERRRGKGHDGLYQGLSTATKDTYDALHMQTLAPR
GSGATNFSLLKQAGDVEENPGPMSNTVLADSAWGITLLSWVTVFLLGTSSADSGVVQSPRHIIKEKGGRSVLTCIPISGHSNVVWYQQTL
GKELKFLIQHYEKVERDKGFLPSRFSVQQFDDYHSEMNMSALELEDSAMYFCASSRANYEQYFGPGTRLTVLCKIEFMYPPPYLDNERSN
GTIIHIKEKHLCHTQSSPKLFWALVVVAGVLFCYGLLVTVALCVIWTNSRRNRLLQSDYMNMTPRRPGLTRKPYQPYAPARDFAAYRPRA
KFSRSAETAANLQDPNQLYNELNLGRREEYDVLEKKRARDPEMGGKQQRRRNPQEGVYNALQKDKMAEAYSEIGTKGERRRGKGHDGLYQ
GLSTATKDTYDALHMQTLAPRGSGEGRGSLLTCGDVEENPGPMGNNCYNVVVIVLLLVGCEKVGAVQNSCDNCQPGTFCRKYNPVCKSCP
PSTFSSIGGQPNCNICRVCAGYFRFKKFCSSTHNAECECIEGFHCLGPQCTRCEKDCRPGQELTKQGCKTCSLGTVNDQNGTGVCRPWTN
CSLDGRSVLKTGTTEKDVVCGPPVVSFSPSTTISVTPEGGPGGHSLQVLTLFLALTSALLLALIFTLLFSVLKWIRKKFPHIFKQPFKKT
TGAAQEEDACSCRCPQEEEGGGGGYEL* (SEQ ID NO: 41)
Nucleotides chosen to be part of the most diverse CD28:CD3ζ region
TGTAAGATCGAGTTTATGTATCCACCACCATACCTCGATAACGAACGCTCCAATGGAACCATCATACATATCAAAGAGAAGCATCTCTGC
CATACACAATCCTCCCCTAAATTGTTCTGGGCCCTGGTTGTCGTGGCCGGAGTCCTCTTTTGCTACGGCTTGCTCGTCACCGTGGCCCTC
TGTGTGATTTGGACAAATAGCCGGCGCAACCGGTTGTTGCAAAGTGACTATATGAATATGACCCCAAGACGCCCTGGGCTCACCAGAAAG
CCTTATCAACCGTATGCTCCCGCCCGCGATTTTGCCGCTTATAGGCCAAGGGCTAAATTCAGCAGATCCGCTGAAACAGCCGCTAACTTG
CAAGATCCTAACCAATTGTACAATGAATTGAACCTCGGCCGGCGCGAAGAATACGATGTCTTGGAAAAGAAAAGAGCCAGGGACCCTGAG
ATGGGAGGCAAGCAACAGAGAAGACGCAATCCCCAAGAGGGGGTGTATAACGCCTTGCAGAAGGACAAGATGGCTGAAGCCTATAGTGAA
ATTGGAACAAAAGGCGAACGGAGGAGAGGCAAAGGCCACGACGGACTCTATCAAGGATTGTCAACCGCAACAAAAGACACATATGATGCA
CTCCATATGCAGACCCTGGCACCGCGGGGAAGCGGC (SEQ ID NO: 42)

gBlocks & Primers

The intracellular portion of the ζ segment will be fused to the C-terminal of the transmembrane portion of CD28 (Maher et al., 2002). Codon optimization for expression in Mus musculus was performed at the IDTDNA website (https://www.idtdna.com/CodonOpt).

| | |
|---|---|
| 05a-AN27-gBLOCK1- | CCTCCCTTTATCCAGCCCTCACTCCTTCTCTAGGCGCCGGAATTCAGATCTACCATGGATAAGATACTGACCGCA |
| aP2A | AGCTTTCTGCTGCTGGGACTGCACCTGGCCGGCGTGAATGGTCAACAGCAGGAAAAGAGAGATCAACAGCAGGTT |
| | CGGCAGTCACCACAATCACTGACAGTTTGGGAGGGAGAAACCGCTATTTTGAACTGCTCATATGAGGATTCCACT |
| | TTCAACTATTTCCCCTGGTACCAACAGTTCCCCGGAGAGGGACCCGCCTTGCTGATTTCCATACGGAGTGTGTCC |
| | GATAAAAAGGAGGACGGCAGGTTCACCATCTTTTTCAACAAGCGAGAAAAAAAGCTGAGCCTCCATATCACCGAC |
| | TCCCAGCCTGGGGATTCAGCTACCTATTTCTGTGCCGCCTCAGACAACTACCAGCTCATCTGGGGATCAGGGACG |
| | AAACTGATTATCAAGCCCGATTGTAAGATCGAGTTTATGTATCCACCACCATACCTCGATAACGAACGCTCCAAT |
| | GGAACCATCATACATATCAAAGAGAAGCATCTCTGCCATACACAATCCTCCCCTAAATTGTTCTGGGCCCTGGTT |
| | GTCGTGGCCGGAGTCCTCTTTTGCTACGGCTTGCTCGTCACCGTGGCCCTCTGTGTGATTTGGACAAATAGCCGG |
| | CGCAACCGGTTGTTGCAAAGTGACTATATGAATATGACCCCAAGACGCCCTGGGCTCACCAGAAAGCCTTATCAA |
| | CCGTATGCTCCCGCCCGCGATTTTGCCGCTTATAGGCCAAGGGCTAAATTCAGCAGATCCGCTGAAACAGCCGCT |
| | AACTTGCAAGATCCTAACCAATTGTACAATGAATTGAACCTCGGCCGGCGCGAAGAATACGATGTCTTGGAAAAG |
| | AAAAGAGCCAGGGACCCTGAGATGGGAGGCAAGCAACAGAGAAGACGCAATCCCCAAGAGGGGGTGTATAACGCC |
| | TTGCAGAAGGACAAGATGGCTGAAGCCTATAGTGAAATTGGAACAAAAGGCGAACGGAGGAGAGGCAAAGGCCAC |
| | GACGGACTCTATCAAGGATTGTCAACCGCAACAAAAGACACATATGATGCACTCCATATGCAGACCCTGGCACCG |
| | CGGGGAAGCGGCGCTACCAATTTTTCCCTGCTGAAGCAAGCCGGCGACGTGGAAGAGAATCCTGGCCCAATGAGT |
| | AACACTGTTCTGGCTGACAGCGCCTGGGGCATTACTCTTCTGTCCTGGGTCACCGTATTCTTGCTGGGCACATCT |
| | AGTGCCGACTCTGGGGTGGTTCAGTCCCCACGACACATCATCAAGGAAAAGGGCGGGCGAAGCGTCCTCACCTGT |
| | ATTCCAATTAGCGGCCATTCCAACGTCGTGTGGTACCAACAGACTTTGGGAAAGGAACTTAAATTTCTGATACAG |
| | CACTATGAGAAGGTAGAGAGAGACAAGGGCTTTCTCCCTTCTAGGTTTTCCGTGCAACAGTTCGACGATTATCAC |
| | ATCAGAGATGAACATGAGCGCACTGGAGCTGGAAGACTCTGCCATGTACTTnGCGCATCAAGTAGAGCTAATTAC |
| | GAGCAATACTTCGGACCTGGGACACGCCTTACCGTCCTCTGCAAAATAGAATTCATGTACCCTCCGCCTTATTTG |
| | GATAACGAGAGGAGTAACGGCACAATTATCCACATTAGGAAAAACACCTGTGTCACACCCAGAGTAGCCCCAAGC |
| | TGTTTTGGGCACTTGTGG |
| 05b-AN28-gBLOCK2- | CCTGTGTCACACCCAGAGTAGCCCCAAGCTGTTTTGGGCACTTGTGGTGGTCGCAGGCGTGCTGTTCTGTTATGG |
| bT2A41BBL | GCTGTTGGTGACGGTTGCACTGTGCGTAATCTGGACCAACTCTAGAAGGAATAGACTGCTCCAGTCCGATTACAT |
| | GAACATGACTCCTCGCAGGCCGGGACTGACTCGCAAACCGTACCAGCCCTACGCCCCTGCTAGAGACTTCGCAGC |
| | CTACCGCCCCCGCGCCAAGTTTTCACGCAGTGCCGAGACTGCTGCCAATCTGCAGGACCCCAATCAGCTCTATA |
| | ACGAGCTTAATCTGGGAAGAAGGGAGGAGTATGACGTGCTTGAGAAAAAGCGGGCTCGCGATCCAGAAATGGGCG |
| | GGAAACAGCAACGGCGCAGGAACCCTCAGGAAGGCGTTTACAATGCTCTTCAAAAAGATAAAATGGCCGAGGCAT |
| | ACTCCGAGATCGGCACCAAGGGAGAGAGAAGACGGGGAAAGGGACATGATGGCTTGTACCAGGGGCTCAGCACA |
| | GCTACCAAGGATACCTACGACGCTCTGCACATGCAAACACTCGCTCCCAGAGGGTCAGGGGAAGGCCGGGGAAG |
| | TCTGCTCACATGTGGCGACGTGGAGGAGAATCCCGGGCCCATGGGTAATAACTGCTATAACGTGGTCGTGATCG |
| | TCTTGCTGCTCGTGGGATGTGAGAAGGTGGGAGCTGTGCAGAACAGTTGCGACAACTGTCAACCTGGTACTTTT |
| | TGCGAGATGCCGGAAATACAACCCCGTCTGCAAGAGTTGTCCTCCAAGTACATTTTCATCTATCGGGGGCCAGCC |
| | TAACTGTAATATCTGCAGGGTGTGTGCTGGGTATTTCCGCTTTAAGAAGTTTTGTTCCTCAACACACAACGCTGA |
| | GTGCGAGTGTATCGAGGGATTCCATTGTCTGGGGCCCCAGTGCACTAGGAGGATTGTCGCCCAGGGCAAGAACT |
| | CACAAAGCAGGGCTGTAAGACGTGTTCTTTGGGCACCTTCAATGATCAGAACGGAACAGGGGTCTGCCGACCTTG |
| | GACCAACTGTAGTCTCGATGGGCGCTCTGTGCTCAAGACAGGCACGACCGAGAAAGACGTGGTGTGTGGACCACC |
| | TGTGGTTTCCTTCTCCCCTTCTACAACCATTTCCGTTACCCCTGAAGGAGGGCCCGGCGGCCACTCTCTGCAAGT |
| | GCTCACTCTGTTTTTGGCCCTCACCAGTGCTCTGTTGCTCGCCCTGATCTTCATCACACTGCTGTTCAGTGTGCT |
| | GAAGTGGATCAGAAAGAAGTTCCCACATATCTTCAAGCAACCTTTCAAAAAGACCACGGGAGCAGCCCAGGAGGA |
| | GGACGCTTGTTCATGCAGATGCCCCCAGGAGGAAGAGGGAGGTGGCGGTGGATATGAATTGTAAGGATCCCAATT |
| | GCTCGAGGATCAATTCCGCCCCTCTCCCTCC |
| 05c-AN29-gBLOCK3- | TGTAAGATCGAGTTTATGTATCCACCACCATACCTCGATAACGAACGCTCCAATGGAACCATCATACATATCAA |
| 28zP2A | AGAGAAGCATCTCTGCCATACACAATCCTCCCCTAAATTGTTCTGGGCCCTGGTTGTCGTGGCCGGAGTCCTCT |
| | TTTGCTACGGCTTGCTCGTCACCGTGGCCCTCTGTGTGATTTGGACAAATAGCCGGCGCAACCGGTTGTTGCAA |
| | AGTGACTATATGAATATGACCCCAAGACGCCCTGGGCTCACCAGAAAGCCTTATCAACCGTATGCTCCCGCCCG |
| | CGATTTTGCCGCTTATAGGCCAAGGGCTAAATTCAGCAGATCCGCTGAAACAGCCGCTAACTTGCAAGATCCTA |
| | ACCAATTGTACAATGAATTGAACCTCGGCCGGCGCGAAGAATACGATGTCTTGGAAAAGAAAAGAGCCAGGGACC |
| | CTGAGATGGGAGGCAAGCAACAGAGAAGACGCAATCCCCAAGAGGGGGTGTATAACGCCTTGCAGAAGGACAAG |
| | ATGGCTGAAGCCTATAGTGAAATTGGAACAAAAGGCGAACGGAGGAGAGGCAAAGGCCACGACGGACTCTATC |
| | AAGGATTGTCAACCGCAACAAAAGACACATATGATGCACTCCATATGCAGACCCTGGCACCGCGGGGAAGCGGC |
| | GCTACCAATTTTTCCCTGCTGAAGCAAGCCGGCGACGTGGAAGAGAATCCTGGCCCA |
| 06a-AN30-GibVec | GGTAGATCTGAATTCCGGCGCCTAGAGAAGG |
| RV-PREinsert | |
| 06b-AN31-GibVec | GGATCCCAATTGCTCGAGGATCAATTCCG |
| FW-POSTinsert | |
| 06c-AN32-GibVec | TGCAAAATAGAATTCATGTACCCTCCGCCTTATTTGG |
| FW-latePOSTinsert | |
| 07a-AN33-SEQP_1 | CCTCCGCCTCCTCTTCCTCCATCC |
| 07b-AN34-SEQP_2 | CAAAGAGAAGCATCTCTGCCATACACAATCCTCC |
| 07c-AN35-SEQP_3 | GCTACCAATTTTTCCCTGCTGAAGCAAGC |
| 07d-AN36-SEQP_4 | GCTGTTCTGTTATGGGCTGTTGGTGACG |
| 07e-AN37-SEQP_5 | GGATGTGAGAAGGTGGGAGCTGTGC |
| 07f-AN38-SEQP_6 | GCAACCTTTCAAAAAGACCACGGGAGC |

### Other Embodiments

While the invention has been described in conjunction with the detailed description thereof; the foregoing description is intended to illustrate and not limit the scope of the invention, which is defined by the scope of the appended claims. Other aspects, advantages, and modifications are within the scope of the following claims.

1. A chimeric antigen receptor (CAR) for use in treating a subject in need thereof comprising an intracellular signaling domain, a transmembrane domain and an extracellular domain comprising i) the variable region of a T-cell receptor specific for a tumor-associated antigen or ii) the variable region of a T-cell receptor specific for a self-antigen, wherein said T-cell receptor is derived from a T-lymphocyte obtained from said subject.

2. The CAR of claim 1, wherein the transmembrane domain further comprises a stalk region positioned between the extracellular domain and the transmembrane domain.

3. The CAR of claim 1, wherein the transmembrane domain comprises CD28.

4. The CAR of claim 1, further comprising one or more additional costimulatory molecules positioned between the transmembrane domain and the intracellular signaling domain.

5. The CAR of claim 4, wherein the costimulatory molecules is CD28, 4-1BB, 4-1BBL ICOS, or OX40.

6. The CAR of claim 1, wherein the intracellular signaling domain comprises a CD3 zeta chain.

7. A nucleic acid encoding the CAR of claim 1.

8. A vector comprising the nucleic acid claim 7.

9. A cell comprising the vector of claim 8.

10. The cell of claim 9, wherein the cell is a T-cell.

11. The cell of claim 10, wherein the T-cell is a CD4+ T-cell and/or CD8+ T-cell; or wherein the T-cell is a T regulatory cell (Treg) or a T follicular regulatory cell (TFR).

12. (canceled)

13. A genetically engineered cell which expresses and bears on the cell surface membrane the chimeric antigen receptor of claim 1.

14. The cell of claim 13, wherein the cell is a T-cell.

15. The cell of claim 14, wherein the T-cell is a CD4⁺ or CD8⁺ T-cell; or wherein the T-cell is a T regulatory cell (Treg) or a T follicular regulatory cell (TFR).

16. (canceled)

17. A pharmaceutical composition comprising a population of the genetically engineered cell of claim 13.

18. A method of treating cancer in a subject in need thereof comprising administering the composition of claim 17, wherein the extracellular domain of the CAR is a variable region of a T-cell receptor specific for a tumor-associated antigen derived from the subject.

19. A method of treating or preventing an autoimmune disorder in a subject in need thereof comprising administering the composition of claim 17, wherein the extracellular domain of the CAR is a variable region of a T-cell receptor specific for a self-antigen derived from the subject; or
a method of treating or preventing graft rejection in a subject in need thereof comprising administering the composition of claim 17, wherein the extracellular domain of the CAR is a variable region of a T-cell receptor specific for a self-antigen derived from the graft tissue.

20. (canceled)

21. A method of isolating a subject specific neoantigen comprising:
a) isolating a plurality of class I and class H MHC peptides from a population of cells obtained from said subject;
b) contacting the plurality of peptide isolated from step (a) with a T-cell receptor (TCR) multimer, wherein said TCR was isolated from a single T-cell isolated from said subject to form an TCR multimer-MHC peptide complex;
c) isolating the MHC peptide from said complex.

22. The method of claim 21, further comprising sequencing said isolated MHC peptide to identify said peptide; or
wherein said isolation of the plurality of peptide is immunochemically; or
wherein said T-cell is a tumor infiltrating lymphocyte, lymphocyte isolated from a transplanted organ or a lymphocyte isolated from an autoimmune site.

23.-24. (canceled)

25. A neoantigenic peptide identified by the method of claim 22; or a vaccine composition comprising the peptide identified in the method of claim 22.

26. (canceled)


SEQUENCE LISTING (54 sequences)

SEQ ID NO: 1 - protein, 28 aa, Artificial Sequence
  Synthetic Polynucleotide
MDKILTASFLLLGLHLAGVNGQQQEKRD

SEQ ID NO: 2 - protein, 93 aa, Artificial Sequence
  Synthetic Polynucleotide
QQQVRQSPQSLTVWEGETAILNCSYEDSTFNYFPWYQQFPGEGPALLISIRSVSDKKEDGRFTIFFNKREKKLSLHITDSQPGDSATYFCAAS

SEQ ID NO: 3 - protein, 135 aa, Artificial Sequence
  Synthetic Polynucleotide
DIQNPEPAVYQLKDPRSQDSTLCLFTDFDSQINVPKTMESGTFITDKTVLDMEAMDSKSNGAIAWSNQTSFTCQDIFKETNATYPSSDVPCDATLTEKSFETDMNLNFQNLSVMGLRILLLKVAGFNLLMTLRLW

SEQ ID NO: 4 - protein, 19 aa, Artificial Sequence
  Synthetic Polynucleotide
ATNFSLLKQAGDVEENPGP

SEQ ID NO: 5 - protein, 29 aa, Artificial Sequence
  Synthetic Polynucleotide
MSNTVLADSAWGITLLSWVTVFLLGTSSA

SEQ ID NO: 6 - protein, 94 aa, Artificial Sequence
  Synthetic Polynucleotide
DSGVVQSPRHIIKEKGGRSVLTCIPISGHSNVVWYQQTLGKELKFLIQHYEKVERDKGFLPSRFSVQQFDDYHSEMNMSALELEDSAMYFCASS

SEQ ID NO: 7 - protein, 15 aa, Artificial Sequence
  Synthetic Polynucleotide
NYEQYFGPGTRLTVL

SEQ ID NO: 8 - protein, 173 aa, Artificial Sequence
  Synthetic Polynucleotide
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 9 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 10 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 11 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 12 - protein, 164 aa, Mus musculus
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 13 - protein, 256 aa, Mus musculus
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVLTLFLALTSALLLALIFITLLFSVLKWIRKKFPHIFKQPFKKTTGAAQEEDACSCRCPQEEEGGGGGYEL

SEQ ID NO: 14 - protein, 526 aa, Mus musculus
METDAIDGYITCDNELSPEGEHANMAIDLTSSTPNGQHASPSHMTSTNSVKLEMQSDEECDRQPLSREDEIRGHDEGSSLEEPLIESSEVADNRKVQDLQGEGGIRLPNGKLKCDVCGMVCIGPNVLMVHKRSHTGERPFHCNQCGASFTQKGNLLRHIKLHSGEKPFKCPFCSYACRRRDALTGHLRTHSVGKPHKCNYCGRSYKQRSSLEEHKERCHNYLQNVSMEAAGQVMSHHVPPMEDCKEQEPIMDNNISLVPFERPAVIEKLTANMGKRKSSTPQKFVGEKLMRFSYPDIHFDMNLTYEKEAELMQSHMMDQAINNAITYLGAEALHPLMQHAPSTIAEVAPVISSAYSQVYHPNRIERPISRETSDSHENNMDGPISLIRPKSRPQEREASPSNSCLDSTDSESSHDDRQSYQGNPALNPKRKQSPAYMKEDVKALDATKAPKGSLKDIYKVFNGEGEQIRAFKCEHCRVLFLDHVMYTIHMGCHGYRDPLECNICGYRSQDRYEFSSHIVRGEHTFH

SEQ ID NO: 15 - protein, 429 aa, Mus musculus
MPNPRPAKPMAPSLALGPSPGVLPSWKTAPKGSELLGTRGSGGPFQGRDLRSGAHTSSSLNPLPPSQLQLPTVPLVMVAPSGARLGPSPHLQALLQDRPHFMHQLSTVDAHAQTPVLQVRPLDNPAMISLPPPSAATGVFSLKARPGLPPGINVASLEWVSREPALLCTFPRSGTPRKDSNLLAAPQGSYPLLANGVCKWPGCEKVFEEPEEFLKHCQADHLLDEKGKAQCLLQREVVQSLEQQLELEKEKLGAMQAHLAGKMALAKAPSVASMDKSSCCIVATSTQGSVLPAWSAPREAPDGGLFAVRRHLWGSHGNSSFPEFFHNMDYFKYHNMRPPFTYATLIRWAILEAPERQRTLNEIYHWFTRMFAYFRNHPATWKNAIRHNLSLHKCFVRVESEKGAVWTVDEFEFRKKRSQRPNKCSNPCP

SEQ ID NO: 16 - protein, 22 aa, Artificial Sequence
  Synthetic Polynucleotide
GSGATNFSLLKQAGDVEENPGP

SEQ ID NO: 17 - protein, 21 aa, Artificial Sequence
  Synthetic Polynucleotide
GSGEGRGSLLTCGDVEENPGP

SEQ ID NO: 18 - protein, 23 aa, Artificial Sequence
  Synthetic Polynucleotide
GSGQCTNYALLKLAGDVESNPGP

SEQ ID NO: 19 - protein, 28 aa, Artificial Sequence
  Synthetic Polynucleotide
MDKILTASFLLLGLHLAGVNGQQQEKRD

SEQ ID NO: 20 - protein, 93 aa, Artificial Sequence; identical to SEQ ID NO: 2
  Synthetic Polynucleotide

SEQ ID NO: 21 - protein, 17 aa, Artificial Sequence
  Synthetic Polynucleotide
DNYQLIWGSGTKLIIKP

SEQ ID NO: 22 - protein, 135 aa, Artificial Sequence; identical to SEQ ID NO: 3
  Synthetic Polynucleotide

SEQ ID NO: 23 - protein, 19 aa, Artificial Sequence
  Synthetic Polynucleotide
ATNFSLLKQAGDVEENPGP

SEQ ID NO: 24 - protein, 29 aa, Artificial Sequence
  Synthetic Polynucleotide
MSNTVLADSAWGITLLSWVTVFLLGTSSA

SEQ ID NO: 25 - protein, 94 aa, Artificial Sequence; identical to SEQ ID NO: 6
  Synthetic Polynucleotide

SEQ ID NO: 26 - protein, 15 aa, Artificial Sequence
  Synthetic Polynucleotide
NYEQYFGPGTRLTVL

SEQ ID NO: 27 - protein, 173 aa, Artificial Sequence; identical to SEQ ID NO: 8
  Synthetic Polynucleotide

SEQ ID NO: 28 - protein, 28 aa, Artificial Sequence
  Synthetic Polynucleotide
MDKILTASFLLLGLHLAGVNGQQQEKRD

SEQ ID NO: 29 - protein, 20 aa, Artificial Sequence
  Synthetic Polynucleotide
QQQVRQSPQSDSATYFCAAR

SEQ ID NO: 30 - protein, 16 aa, Artificial Sequence
  Synthetic Polynucleotide
GNRIFFGDGTQLVVKP

SEQ ID NO: 31 - protein, 20 aa, Artificial Sequence
  Synthetic Polynucleotide
NIQNPEPAVYNLLMTLRLWS

SEQ ID NO: 32 - protein, 19 aa, Artificial Sequence
  Synthetic Polynucleotide
ATNFSLLKQAGDVEENPGP

SEQ ID NO: 33 - protein, 29 aa, Artificial Sequence
  Synthetic Polynucleotide
MSNTAFPDPAWNTTLLSWVALFLLGTSSA

SEQ ID NO: 34 - protein, 21 aa, Artificial Sequence
  Synthetic Polynucleotide
NSGVVQSPRYISAVYFCASSL

SEQ ID NO: 35 - protein, 16 aa, Artificial Sequence
  Synthetic Polynucleotide
SQNTLYFGAGTRLSVL

SEQ ID NO: 36 - protein, 20 aa, Artificial Sequence
  Synthetic Polynucleotide
EDLRNVTPPKLMAMVKRKNS

SEQ ID NO: 37 - protein, 139 aa, Artificial Sequence
  Synthetic Polynucleotide
MDKILTASFLLLGLHLAGVNGQQQEKRDQQQVRQSPQSLTVWEGETAILNCSYEDSTFNYFPWYQQFPGEGPALLISIRSVSDKKEDGRFTIFFNKREKKLSLHITDSQPGDSATYFCAASDNYQLIWGSGTKLIIKPD

SEQ ID NO: 38 - protein, 241 aa, Artificial Sequence
  Synthetic Polynucleotide
CKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGATNFSLLKQAGDVEENPGP

SEQ ID NO: 39 - protein, 140 aa, Artificial Sequence
  Synthetic Polynucleotide
MSNTVLADSAWGITLLSWVTVFLLGTSSADSGVVQSPRHIIKEKGGRSVLTCIPISGHSNVVWYQQTLGKELKFLIQHYEKVERDKGFLPSRFSVQQFDDYHSEMNMSALELEDSAMYFCASSRANYEQYFGPGTRLTVL

SEQ ID NO: 40 - protein, 496 aa, Artificial Sequence
  Synthetic Polynucleotide
CKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGEGRGSLLTCGDVEENPGPMGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVLTLFLALTSALLLALIFITLLFSVLKWIRKKFPHIFKQPFKKTTGAAQEEDACSCRCPQEEEGGGGGYEL

SEQ ID NO: 41 - protein, 1015 aa, Artificial Sequence
  Synthetic Polynucleotide
MDKILTASFLLLGLHLAGVNGQQQEKRDQQQVRQSPQSLTVWEGETAILNCSYEDSTFNYFPWYQQFPGEGPALLISIRSVSDKKEDGRFTIFFNKREKKLSLHITDSQPGDSATYFCAASDNYQLIWGSGTKLIIKPDCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGATNFSLLKQAGDVEENPGPMSNTVLADSAWGITLLSWVTVFLLGTSSADSGVVQSPRHIIKEKGGRSVLTCIPISGHSNVVWYQQTLGKELKFLIQHYEKVERDKGFLPSRFSVQQFDDYHSEMNMSALELEDSAMYFCASSRANYEQYFGPGTRLTVLCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGEGRGSLLTCGDVEENPGPMGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVLTLFLALTSALLLALIFITLLFSVLKWIRKKFPHIFKQPFKKTTGAAQEEDACSCRCPQEEEGGGGGYE

SEQ ID NO: 42 - protein, 666 aa, Artificial Sequence
  Synthetic Polynucleotide
TGTAAGATCGAGTTTATGTATCCACCACCATACCTCGATAACGAACGCTCCAATGGAACCATCATACATATCAAAGAGAAGCATCTCTGCCATACACAATCCTCCCCTAAATTGTTCTGGGCCCTGGTTGTCGTGGCCGGAGTCCTCTTTTGCTACGGCTTGCTCGTCACCGTGGCCCTCTGTGTGATTTGGACAAATAGCCGGCGCAACCGGTTGTTGCAAAGTGACTATATGAATATGACCCCAAGACGCCCTGGGCTCACCAGAAAGCCTTATCAACCGTATGCTCCCGCCCGCGATTTTGCCGCTTATAGGCCAAGGGCTAAATTCAGCAGATCCGCTGAAACAGCCGCTAACTTGCAAGATCCTAACCAATTGTACAATGAATTGAACCTCGGCCGGCGCGAAGAATACGATGTCTTGGAAAAGAAAAGAGCCAGGGACCCTGAGATGGGAGGCAAGCAACAGAGAAGACGCAATCCCCAAGAGGGGGTGTATAACGCCTTGCAGAAGGACAAGATGGCTGAAGCCTATAGTGAAATTGGAACAAAAGGCGAACGGAGGAGAGGCAAAGGCCACGACGGACTCTATCAAGGATTGTCAACCGCAACAAAAGACACATATGATGCACTCCATATGCAGACCCTGGCACCGCGGGGAAGCGGC

SEQ ID NO: 43 - protein, 1744 aa, Artificial Sequence
  Synthetic Polynucleotide
CCTCCCTTTATCCAGCCCTCACTCCTTCTCTAGGCGCCGGAATTCAGATCTACCATGGATAAGATACTGACCGCAAGCTTTCTGCTGCTGGGACTGCACCTGGCCGGCGTGAATGGTCAACAGCAGGAAAAGAGAGATCAACAGCAGGTTCGGCAGTCACCACAATCACTGACAGTTTGGGAGGGAGAAACCGCTATTTTGAACTGCTCATATGAGGATTCCACTTTCAACTATTTCCCCTGGTACCAACAGTTCCCCGGAGAGGGACCCGCCTTGCTGATTTCCATACGGAGTGTGTCCGATAAAAAGGAGGACGGCAGGTTCACCATCTTTTTCAACAAGCGAGAAAAAAAGCTGAGCCTCCATATCACCGACTCCCAGCCTGGGGATTCAGCTACCTATTTCTGTGCCGCCTCAGACAACTACCAGCTCATCTGGGGATCAGGGACGAAACTGATTATCAAGCCCGATTGTAAGATCGAGTTTATGTATCCACCACCATACCTCGATAACGAACGCTCCAATGGAACCATCATACATATCAAAGAGAAGCATCTCTGCCATACACAATCCTCCCCTAAATTGTTCTGGGCCCTGGTTGTCGTGGCCGGAGTCCTCTTTTGCTACGGCTTGCTCGTCACCGTGGCCCTCTGTGTGATTTGGACAAATAGCCGGCGCAACCGGTTGTTGCAAAGTGACTATATGAATATGACCCCAAGACGCCCTGGGCTCACCAGAAAGCCTTATCAACCGTATGCTCCCGCCCGCGATTTTGCCGCTTATAGGCCAAGGGCTAAATTCAGCAGATCCGCTGAAACAGCCGCTAACTTGCAAGATCCTAACCAATTGTACAATGAATTGAACCTCGGCCGGCGCGAAGAATACGATGTCTTGGAAAAGAAAAGAGCCAGGGACCCTGAGATGGGAGGCAAGCAACAGAGAAGACGCAATCCCCAAGAGGGGGTGTATAACGCCTTGCAGAAGGACAAGATGGCTGAAGCCTATAGTGAAATTGGAACAAAAGGCGAACGGAGGAGAGGCAAAGGCCACGACGGACTCTATCAAGGATTGTCAACCGCAACAAAAGACACATATGATGCACTCCATATGCAGACCCTGGCACCGCGGGGAAGCGGCGCTACCAATTTTTCCCTGCTGAAGCAAGCCGGCGACGTGGAAGAGAATCCTGGCCCAATGAGTAACACTGTTCTGGCTGACAGCGCCTGGGGCATTACTCTTCTGTCCTGGGTCACCGTATTCTTGCTGGGCACATCTAGTGCCGACTCTGGGGTGGTTCAGTCCCCACGACACATCATCAAGGAAAAGGGCGGGCGAAGCGTCCTCACCTGTATTCCAATTAGCGGCCATTCCAACGTCGTGTGGTACCAACAGACTTTGGGAAAGGAACTTAAATTTCTGATACAGCACTATGAGAAGGTAGAGAGAGACAAGGGCTTTCTCCCTTCTAGGTTTTCCGTGCAACAGTTCGACGATTATCACTCAGAGATGAACATGAGCGCACTGGAGCTGGAAGACTCTGCCATGTACTTTTGCGCATCAAGTAGAGCTAATTACGAGCAATACTTCGGACCTGGGACACGCCTTACCGTCCTCTGCAAAATAGAATTCATGTACCCTCCGCCTTATTTGGATAACGAGAGGAGTAACGGCACAATTATCCACATTAAGGAAAAACACCTGTGTCACACCCAGAGTAGCCCCAAGCTGTTTTGGGCACTTGTGG

SEQ ID NO: 44 - protein, 1450 aa, Artificial Sequence
  Synthetic Polynucleotide
CCTGTGTCACACCCAGAGTAGCCCCAAGCTGTTTTGGGCACTTGTGGTGGTCGCAGGCGTGCTGTTCTGTTATGGGCTGTTGGTGACGGTTGCACTGTGCGTAATCTGGACCAACTCTAGAAGGAATAGACTGCTCCAGTCCGATTACATGAACATGACTCCTCGCAGGCCGGGACTGACTCGCAAACCGTACCAGCCCTACGCCCCTGCTAGAGACTTCGCAGCCTACCGCCCCCGCGCCAAGTTTTCACGCAGTGCCGAGACTGCTGCCAATCTGCAGGACCCCAATCAGCTCTATAACGAGCTTAATCTGGGAAGAAGGGAGGAGTATGACGTGCTTGAGAAAAAGCGGGCTCGCGATCCAGAAATGGGCGGGAAACAGCAACGGCGCAGGAACCCTCAGGAAGGCGTTTACAATGCTCTTCAAAAAGATAAAATGGCCGAGGCATACTCCGAGATCGGCACCAAGGGAGAGAGAAGACGGGGAAAGGGACATGATGGCTTGTACCAGGGGCTCAGCACAGCTACCAAGGATACCTACGACGCTCTGCACATGCAAACACTCGCTCCCAGAGGGTCAGGGGAAGGCCGGGGAAGTCTGCTCACATGTGGCGACGTGGAGGAGAATCCCGGGCCCATGGGTAATAACTGCTATAACGTGGTCGTGATCGTCTTGCTGCTCGTGGGATGTGAGAAGGTGGGAGCTGTGCAGAACAGTTGCGACAACTGTCAACCTGGTACTTTTTGCCGGAAATACAACCCCGTCTGCAAGAGTTGTCCTCCAAGTACATTTTCATCTATCGGGGGCCAGCCTAACTGTAATATCTGCAGGGTGTGTGCTGGGTATTTCCGCTTTAAGAAGTTTTGTTCCTCAACACACAACGCTGAGTGCGAGTGTATCGAGGGATTCCATTGTCTGGGGCCCCAGTGCACTAGGTGCGAGAAGGATTGTCGCCCAGGGCAAGAACTCACAAAGCAGGGCTGTAAGACGTGTTCTTTGGGCACCTTCAATGATCAGAACGGAACAGGGGTCTGCCGACCTTGGACCAACTGTAGTCTCGATGGGCGCTCTGTGCTCAAGACAGGCACGACCGAGAAAGACGTGGTGTGTGGACCACCTGTGGTTTCCTTCTCCCCTTCTACAACCATTTCCGTTACCCCTGAAGGAGGGCCCGGCGGCCACTCTCTGCAAGTGCTCACTCTGTTTTTGGCCCTCACCAGTGCTCTGTTGCTCGCCCTGATCTTCATCACACTGCTGTTCAGTGTGCTGAAGTGGATCAGAAAGAAGTTCCCACATATCTTCAAGCAACCTTTCAAAAAGACCACGGGAGCAGCCCAGGAGGAGGACGCTTGTTCATGCAGATGCCCCCAGGAGGAAGAGGGAGGTGGCGGTGGATATGAATTGTAAGGATCCCAATTGCTCGAGGATCAATTCCGCCCCTCTCCCTCC

SEQ ID NO: 45 - protein, 723 aa, Artificial Sequence
  Synthetic Polynucleotide
TGTAAGATCGAGTTTATGTATCCACCACCATACCTCGATAACGAACGCTCCAATGGAACCATCATACATATCAAAGAGAAGCATCTCTGCCATACACAATCCTCCCCTAAATTGTTCTGGGCCCTGGTTGTCGTGGCCGGAGTCCTCTTTTGCTACGGCTTGCTCGTCACCGTGGCCCTCTGTGTGATTTGGACAAATAGCCGGCGCAACCGGTTGTTGCAAAGTGACTATATGAATATGACCCCAAGACGCCCTGGGCTCACCAGAAAGCCTTATCAACCGTATGCTCCCGCCCGCGATTTTGCCGCTTATAGGCCAAGGGCTAAATTCAGCAGATCCGCTGAAACAGCCGCTAACTTGCAAGATCCTAACCAATTGTACAATGAATTGAACCTCGGCCGGCGCGAAGAATACGATGTCTTGGAAAAGAAAAGAGCCAGGGACCCTGAGATGGGAGGCAAGCAACAGAGAAGACGCAATCCCCAAGAGGGGGTGTATAACGCCTTGCAGAAGGACAAGATGGCTGAAGCCTATAGTGAAATTGGAACAAAAGGCGAACGGAGGAGAGGCAAAGGCCACGACGGACTCTATCAAGGATTGTCAACCGCAACAAAAGACACATATGATGCACTCCATATGCAGACCCTGGCACCGCGGGGAAGCGGCGCTACCAATTTTTCCCTGCTGAAGCAAGCCGGCGACGTGGAAGAGAATCCTGGCCCA

SEQ ID NO: 46 - protein, 31 aa, Artificial Sequence
  Synthetic Polynucleotide
GGTAGATCTGAATTCCGGCGCCTAGAGAAGG

SEQ ID NO: 47 - protein, 29 aa, Artificial Sequence
  Synthetic Polynucleotide
GGATCCCAATTGCTCGAGGATCAATTCCG

SEQ ID NO: 48 - protein, 37 aa, Artificial Sequence
  Synthetic Polynucleotide
TGCAAAATAGAATTCATGTACCCTCCGCCTTATTTGG

SEQ ID NO: 49 - protein, 24 aa, Artificial Sequence
  Synthetic Polynucleotide
CCTCCGCCTCCTCTTCCTCCATCC

SEQ ID NO: 50 - protein, 34 aa, Artificial Sequence
  Synthetic Polynucleotide
CAAAGAGAAGCATCTCTGCCATACACAATCCTCC

SEQ ID NO: 51 - protein, 29 aa, Artificial Sequence
  Synthetic Polynucleotide
GCTACCAATTTTTCCCTGCTGAAGCAAGC

SEQ ID NO: 52 - protein, 28 aa, Artificial Sequence
  Synthetic Polynucleotide
GCTGTTCTGTTATGGGCTGTTGGTGACG

SEQ ID NO: 53 - protein, 25 aa, Artificial Sequence
  Synthetic Polynucleotide
GGATGTGAGAAGGTGGGAGCTGTGC

SEQ ID NO: 54 - protein, 27 aa, Artificial Sequence
  Synthetic Polynucleotide
GCAACCTTTCAAAAAGACCACGGGAGC